(19) United States
(12) Patent Application Publication
(10) Pub. No.: US 2025/0349433 A1
(43) Pub. Date: Nov. 13, 2025

(54) METHODS AND SYSTEMS FOR DETERMINING DENTAL CARIES

(71) Applicant: J. Craig Venter Institute, Inc., La Jolla, CA (US)

(72) Inventors: Chris L. Dupont, La Jolla, CA (US); Josh L. Espinoza, La Jolla, CA (US)

(21) Appl. No.: 18/855,586

(22) PCT Filed: May 9, 2023
(86) PCT No.: PCT/US2023/021575

§ 371 Date: Oct. 9, 2024

Related U.S. Application Data
(60) Provisional application No. 63/340,774, filed on May 11, 2022.

(51) Int. Cl. G16H 50/30; G16B 30/10; G16B 40/30

(52) U.S. Cl. CPC G16H 50/30; G16B 30/10; G16B 40/30

(57) ABSTRACT

A sequencing module configured to provide metatranscriptomic reads from an oral sample from a subject; metatranscriptomic reads from the sequencing of oral sample and identify and cluster microbes identified in the oral sample into taxon clusters (TCs) using the metatranscriptomic reads mapped to a metagenomic library; generate TC-specific orthogroups for each of the TCs via protein clustering; determine KEGG orthology for each of the TC-specific orthogroups, or genes directly; generate phylogenomic functional categories (PGFCs) from grouping of gene expression counts by the KEGG modules for each of the TCs; retain the PGFCs having an MCR above an MCR threshold to obtain input data; and identify or predict, using a classifier model including variables selected by a feature selection machine learning algorithm, dental caries in said subject based on the input data.

### STATEMENT REGARDING FEDERALLY SPONSORED R&D

This invention was made in part with government support under National Institute of Dental and Craniofacial Research of the National Institutes of Health under award number R01DE019665.

### BACKGROUND

The methods and systems described herein relate to the field of oral health, and, in particular, to determining caries risk, predicting caries, and providing an overall prognosis for cariogenesis. The disclosure further relates to methods of ascertaining caries risk or caries prognosis using metagenomics and metatranscriptomics analysis of the oral microbiome via machine learning techniques, and systems for executing said methods.

Oral diseases, such as dental caries, are a critical concern for public health. Untreated tooth decay is the most common chronic health condition and affects nearly 3.5 billion people worldwide (1). Tooth decay is most common in younger individuals with a prevalence of 20% of children aged 5 to 11 and 13% in adolescents aged 12 to 19, with low-income children being twice as likely to have cavities (2). In most low- and middle-income countries, the prevalence of oral diseases increases with urbanization and inadequate access to medical treatment (3). In high-income countries, dental treatment averages 5% of total health expenditure and 20% of out-of-pocket health expenditure (4) making the disease a socioeconomic issue for all. This silent epidemic has long been coupled with the rise of civilization as early evidence from the Pleistocene era suggests that agriculture and the exploitation of starchy plant foods have burdened mankind with carious lesions since early prehistory (5).

In the modern “ecological plaque hypothesis”, oral diseases arise from environmental perturbations leading to a shift in the endogenous microbial community (6) where the selection of pathogenic bacteria is coupled with the environment and any species with germane traits can contribute to pathogenesis (7). The evidence for this theory is largely from the advent of next generation sequencing (NGS) technologies and the ability to sequence uncultivated organisms. In the context of carious lesions, the environmental perturbation arises from persistent consumption of dietary sugars leading to a decrease in pH and, when sustained, shifts in the population to a more aciduric and cariogenic microbial community, which degrades the enamel (8, 9). Changing environments (e.g., a substantial increase in acidity) can destabilize previously stable microbial communities reconfiguring them into new stability domains, referred to as regime shifts (10), such as a cariogenic microbiome (11). Furthermore, this regime shift of the oral microbiome towards a cariogenic state is the result of an environment partially created by the bacteria themselves creating a complex feed-forward loop. This complex feed-forward loop makes it difficult to diagnose the exact cause of each case and the development of therapeutics for severe cases.

Understanding the roles of microbes in the context of caries-related dysbiosis (from the perspective of human health and not microbial stability) is non-trivial and is often explored using association networks. Association networks such as co-expression (transcriptomics) or co-abundance (genomics) are powerful frameworks for investigating inferred biological interactions by grouping biological features, such as genes or microbes, with related metabolism (12) or complementary ecological niches (13). Despite widescale usage, many approaches do not address NGS compositionality and this is major concern because non-compositionally aware metrics (e.g., correlation) are known to yield spurious associations with no biological meaning (14), which are difficult to use for predicting such conditions in subjects and providing actionable treatment options and plans. Although packages such as WGCNA (15) introduced intuitive and clever ways for analyzing fully-connected weighted gene association networks, they do not support compositionally-aware association metrics such as proportionality (14, 16, 17); thus, the findings using such methods are based on a statistical fallacy. However, awareness of compositional data analysis (CoDA) has increasingly made its way from geology to bioinformatics (14, 16, 18, 19) with many advancements in the context of network analysis (20).

Association networks are often applied to individual organisms in controlled settings (21) and extending these concepts to ecosystems introduce many challenges that arise from the complexity of the data where the exact abundances of biological features are often unknown a priori and the number of features increase by several orders of magnitude. Furthermore, as systems biology deals with interactions amongst biological features, the number of pairwise interactions scale quadratically. The vast number of variables in a microbiome not only makes hypothesis testing difficult but can also lead to statistical artifacts in downstream analysis due to the “curse-of-dimensionality” making interpretation exceedingly difficult (22). Many dimensionality-reduction methods such as PCA, [N]MDS, t-SNE (23), or UMAP (24) lose accessibility to original biological features rendering interpretation limited and unintuitive. The genome-resolved hierarchical complexity of microbiomes result in dynamic distributions of expression or abundance influenced by other microbes and latent environmental variables not accounted for by the experimental design. These community-level datasets require representations of the data that account for these abstractions and group genes within their genome-resolved structure; that is, explainable biological feature engineering. The use of biological feature engineering in this context not only yielded results with biological meaning but also provided predictions of disease states, particularly oral diseases for a particular subject, of >99% accuracy, which may be leveraged into highly tailored treatment plans for the subject.

### SUMMARY

Some of the embodiments described herein relate to systems of determining the oral health of a subject. In one aspect, said systems comprise a sequencing module configured to provide metatranscriptomic reads from an oral sample from a subject; one or more processors in communication with the sequencing module; and a memory in communication with the one or more processors. In some embodiments, the memory stores instructions that, when executed by the one or more processors, the instructions cause the one or more processors to at least: communicate with the sequencing module, such as sending instructions to transfer the metatranscriptomic reads from the sequencing module to the processor; process instructions to read a metagenomic library from a database; identify and cluster microbes identified in the oral sample into taxon clusters (TCs) using the metatranscriptomic reads mapped to the metagenomic library; generate TC-specific orthogroups for each of the TCs via protein clustering; determine KEGG orthology for each of the TC-specific orthogroups, or genes directly, using the metatranscriptomic reads mapped to the TC-specific orthogroups to provide KEGG modules for each of the TCs; generate phylogenomic functional categories (PGFCs) from grouping of gene expression counts by the KEGG modules for each of the TCs; determine a module completion ratio (MCR) for each of the KEGG modules in the PGFCs; retain the PGFCs having the MCR above an MCR threshold to obtain input data, identify or predict, using a feature selection machine learning algorithm, dental caries in said subject based on the input data. In some embodiments, the feature selection machine learning algorithm uses a classifier model including variables selected by the machine learning algorithm. In some embodiments, the instructions stored in the memory further causes the one or more processors to at least to generate a score based on an expression of genes from the metatranscriptomic reads as compared to genes within the KEGG module. In some embodiments, the instructions, when executed by the one or more processors, further causes the one or more processors to predict or provide a diagnosis of dental caries in said subject when the score exceeds a threshold value. the instructions, when executed by the one or more processors, further causes the one or more processors to provide said subject a therapy for dental caries, such as removal of the dental caries, administration of dental fillings, providing a crown, root canal, or extraction.

In some embodiments, the instructions, when executed by the one or more processors, further causes the one or more processors to output a dental care protocol or therapy for said subject, wherein the dental care protocol or therapy differs for subjects that have a score above the threshold value from subjects that have a score below the threshold value.

In some embodiments, the sequencing module comprises a sequencer having an interface for receiving nucleic acids obtained from said oral sample and possessing circuitry configured to sequence said nucleic acids. In some embodiments, cDNA is generated from RNA obtained from said oral sample and said cDNA is sequenced or partially sequenced to yield the metatranscriptomic reads. In some embodiments, the oral sample comprises dental plaque. In some embodiments, the oral sample comprises microbes such as bacteria, or virus, or nucleic acids from bacteria or virus. In some embodiments, the oral sample comprises Abiotrophia, Actinonces, Aggregatibacter, Capnocytophaga, Cardiobacteriun, Corynebacterium, Fusobacterium, Gemella, Granulicatella, Weeksellaceae, Kingella, Lachnoanaerobaculum, Leptotrichia, Neisseria, Prevotella, Rothia, Rodentibacter, or Streptococcus, or any combination thereof. In some embodiments, the oral sample comprises Neisseria and Bacteroidota or nucleic acids therefrom when the oral sample is from a subject lacking dental caries, such as a subject having a score that is less than the threshold value and/or, wherein the oral sample comprises Neisseria and Haemophilus_D parainfluenzae or nucleic acids therefrom when the oral sample is from a subject having dental caries, such as a subject having a score that is above the threshold value.

In some embodiments, the processor further comprises a network module, which is configured to connect to a cloud-based server. In some embodiments, the database is stored on the cloud-based server. In some embodiments, the sequencing module comprises a network unit, which is configured to connect to the cloud-based server. In some embodiments, the sequencing module is configured to communicate with the processor through the cloud-based server.

In some embodiments, the TCs are clustered based on average nucleotide identity (ANI). In some embodiments, a taxonomy of each of the TCs is assigned by a consensus of GTDB-tk classifications, Kraken classifications, and/or a consensus best-hit to existing protein databases. In some embodiments, the metagenomic library comprises a genome catalog for oral plaque obtained from a sex-balanced population. In some embodiments, the feature selection machine learning algorithm comprises differential co-expression analysis of the retained PGFCs. In some embodiments, the differential co-expression analysis builds a differential co-expression network (DCN) from differences between phenotype-specific co-expression networks (PSCNs) using PGFC. In some embodiments, the feature selection machine learning algorithm further comprises hierarchical clustering and differential connectivity analysis to determine clusters of the PGFCs having a differential connectivity above a predictive threshold value for a phenotype. In some embodiments, the phenotype is caries.

Some of the embodiments described herein relate to methods of determining the oral health of a subject. In a second aspect, a method is provided for predicting dental caries in said subject, the method comprising: at a computer system including one or more processors, and memory storing one or more programs for execution by the one or more processors: obtaining, in an electronic format, metatranscriptomic reads for an oral sample of a subject from a sequencing module, wherein the sequencing module comprises a sequencer having an interface for introduction of nucleic acids obtained from said oral sample. In some embodiments, the method further comprises obtaining, in an electronic format, a metagenomic library from a database that is in communication with the computer system. In some embodiments, the method further comprises identifying microbes that are actively expressing genes from the metatranscriptomic reads using the metagenomic library.

In some embodiments, the method further comprises clustering the identified microbes into TCs. In some embodiments, the method further comprises generating TC-specific orthogroups for each of the TCs via protein clustering. In some embodiments, the method further comprises determining KEGG orthology for each of the TC-specific orthogroups, or genes directly, from mapping metatranscriptomic reads to the TC-specific orthogroups to provide KEGG modules for each of the TCs.

In some embodiments, the method further comprises generating PGFCs for each of the TCs by grouping gene expression counts by the KEGG modules for each of the TCs. In some embodiments, the method further comprises determining an MCR for each of the KEGG modules in the PGFCs. In some embodiments, the method further comprises retaining the PGFCs having the MCR above an MCR threshold to obtain input data. In some embodiments, the method further comprises providing the input data into a feature selection machine learning algorithm configured to identify or predict dental caries in said subject from the input data.

In some embodiments, the method further comprises generating a score based on an expression of genes from the metatranscriptomic reads as compared to genes within the KEGG module to predict or provide a diagnosis of dental caries in said subject when the score exceeds a threshold value. In some embodiments, the method further comprises providing said subject a therapy for dental caries, such as removal of the dental caries, administration of dental fillings, providing a crown, root canal, or extraction. In some embodiments, the method further comprises detecting orthogroups in the oral sample relative to a grouping of each of the retained PGFCs; and constructing phenotype-specific co-expression networks (PSCNs) by normalizing counts of the PGFCs with respect to a number of the detected orthogroups within the retained PGFCs.

In some embodiments, the method further comprises performing differential network analysis on the PSCNs to differentiate a caries expression state from a caries-free expression state and to generate differential co-expression networks (DCN) having a differential connectivity value. In some embodiments, the score comprises the differential connectivity value, wherein the threshold value is 0. In some embodiments, a dental caries microbiome is predicted when the differential connectivity value for the DCN is >0. In some embodiments, a dental caries-free microbiome is predicted when the differential connectivity value for the DCN is <0. In some embodiments of the method, the one or more processors output a dental care protocol or therapy for said subject, wherein the dental care protocol or therapy differs for subjects that have a score above the threshold value from subjects that have a score below the threshold value.

In some embodiments, cDNA is generated from RNA obtained from said oral sample and said cDNA is sequenced or partially sequenced to yield the metatranscriptomic reads. In some embodiments, the oral sample comprises dental plaque. In some embodiments, the dental plaque comprises microbes such as bacteria, or virus, or nucleic acids from the bacteria or the virus. In some embodiments, the oral sample comprises Abiotrophia, Actinomyces, Aggregatibacter, Capnocytophaga, Cardiobacterium, Corynebacterium, Fusobacterium, Gemella, Granulicatella, Weeksellaceae, Kingella, Lachnoanaerobaculum, Leptotrichia, Neisseria, Prevotella, Rothia, Rodentibacter, or Streptococcus, or any combination thereof. In some embodiments, the oral sample comprises Neisseria and Bacteroidota or nucleic acids therefrom when the oral sample is from a subject lacking dental caries, such as a subject having a score that is less than the threshold value and/or, wherein the oral sample comprises Neisseria and Haemophilus_D parainfluenzae or nucleic acids therefrom when the oral sample is from a subject having dental caries, such as a subject having a score that is above the threshold value.

In some embodiments, the computer system further comprises a network module, which is configured to connect to a cloud-based server. In some embodiments, the database is stored on the cloud-based server. In some embodiments, the sequencing module is configured to communicate with the computer system through the cloud-based server.

In some embodiments, the TCs are clustered based on ANI. In some embodiments, a taxonomy of each of the TCs is assigned by a consensus of GTDB-tk classifications, Kraken classifications, and/or a consensus best-hit to existing protein databases. In some embodiments, the genomic library comprises a genome catalog for oral plaque obtained from a sex-balanced population.

In a third aspect, a method of predicting dental caries in a subject comprises at a computer system including one or more processors, and memory storing one or more programs for execution by the one or more processors: obtaining, in electronic format, metatranscriptomic reads from a sequencing module, the sequencing module configured to sequence RNA molecules obtained from an oral sample provided by a subject; obtaining, in electronic format, a metagenomic library from a database that is communicative with the computer system; identifying microbes from the metatranscriptomic reads using the metagenomic library; clustering the identified microbes into TCs and determining counts for the TCs; clustering the metatranscriptomic reads into functional categories to generate PGFCs; determining counts for the PGFCs; feeding the counts of the TCs and PGFCs into a machine learning algorithm that processes and sums the counts of the TCs and the PGFCs to generate a score, thereby predicting a diagnosis of dental caries when the score exceeds a threshold value, wherein the functional categories include KEGG modules.

In a fourth aspect, a method of predicting dental caries in a subject comprises at a computer system including one or more processors, and memory storing one or more programs for execution by the one or more processors: obtaining, in electronic format, metatranscriptomic reads from a sequencing module, the sequencing module configured to sequence RNA molecules from an oral sample provided by a subject; obtaining, in electronic format, a metagenomic library from a database that is communicative with the computer system; identifying microbes from the metatranscriptomic reads using the metagenomic library; clustering the identified microbes into TCs and determining counts for the TCs; transforming the TC counts using center log-ratio (CLR) into input data; and feeding the input data into a feature selection machine learning algorithm to identify dental caries from the input data, and optionally, generating a score, thereby predicting a diagnosis of dental caries when the score exceeds a threshold value.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 illustrates a system of predicting dental caries according to some embodiments.

FIG. 2 is a flowchart that illustrates training the machine learning model according to some embodiments.

FIG. 3 is a flowchart that illustrates training the machine learning model according to some embodiments.

FIG. 4 is a flowchart that illustrates training the machine learning model according to some embodiments.

FIG. 5 is a flowchart that illustrates training the machine learning model according to some embodiments.

FIGS. 6A-C depict schematical block diagrams of the metagenome assembled genome pipeline for bacteria and virus according to some embodiments. FIG. 6A depicts a schematical block diagram of prokaryotic assembly and annotation pipelines. FIG. 6B depicts a schematical block diagram of viral assembly and annotation pipelines. FIG. 6C depicts a schematical block diagram of a metagenomic assembled genome pipeline.

FIGS. 7A and 7B depict schematical block diagrams of the genomic feature hierarchy and phylogenomic functional categories respectively according to some embodiments. FIG. 7A depict a schematical block diagram of FastANI clusters and orthogroups. FIG. 7B depict a schematical block diagram of PGFCs showing taxonomic and functional categories created by grouping counts from orthogroups.

FIGS. 8A and 8B show Networks of FastANI clusters for bacterial and viral MAGs respectively according to some embodiments. Network with edge weights corresponding to ANI and nodes representing MAGS, wherein FIG. 8A illustrates bacterial MAGs colored by phylum, and FIG. 8B illustrates viral MAGs colored by either DNA or RNA virus type. Thicker edge weights indicate novel species not found in GTDB-Tk or CheckV for bacterial and viral FastANI clusters, respectively.

FIG. 9 shows taxonomic expression and abundance from clustering using genome-resolved gene expression according to some embodiments.

FIG. 10 shows taxonomic expression:abundance ratios from overlapping metatranscriptomic and metagenomic samples according to some embodiments. Difference in CLR between metatranscriptomics expression (RNA) and metagenomics abundance (DNA) where values indicate higher expression relative to abundance and vice versa.

FIGS. 11A-F show connectivity-based community detection in phenotype-specific coexpression networks (PSCNs) according to some embodiments. FIGS. 11A and 11B show a heatmap of clustered PSCNs for caries and caries-free phenotypes respectively that are sorted by median cluster connectivity [k] in box-plot below with threshold for high-connectivity clusters set at 250 k in both PSCNs. Each i,j value in the heatmap represents the weight of genus i in cluster j divided by the total weight of cluster j (i.e., the weighted proportion of each genus in each cluster). FIGS. 11C and 11D shows a Leiden community detection algorithm applied to high-connectivity PSCN clusters for caries and caries-free phenotypes respectively. Roman numerals indicate PSCN-specific Leiden communities for reference. Pie charts in FIGS. 11C and 11D indicate proportion of genus weight in each Leiden community and colored by phyla. Clustering was performed using the distance version of rho proportionality and Ward linkage. FIG. 11E shows a heatmap of Leiden Community connectivity of FIGS. 11C-D relative to taxonomy. FIG. 11F shows a heatmap of Leiden Community connectivity of FIGS. 11C-D relative to KEGG functional pathways. The heatmaps of FIGS. 11E-F show the connectivity of each grouping relative to the total connectivity in the community.

FIGS. 12A-C show a comparison of PSCNs with respect to taxonomic or functional levels according to some embodiments. FIGS. 12A and 12B show volcano plots of Leiden community PGFCs from FIGS. 11A-F showing a change in scaled connectivity [Δk^{˜}] and −log 2(FDR) to taxonomic and functional PGFCs levels respectively. FIG. 12C shows a sorted barchart of taxa with statistically different connectivities between caries and caries-free PSCNs. FDRs computed using Wilcoxon signed-rank test followed by Benjamini/Hochberg multiple hypothesis correction. Red represents an enrichment in connectivity in PSCN_{Caries} while blue represents an enrichment in connectivity in PSCN_{Caries-free}.

FIGS. 13A-C show differential network analysis between caries and caries-free PSCNs according to some embodiments. FIG. 13A shows a bar chart depicting hierarchical clustering of DCN using Leiden community PGFCs from FIG. 11A-F, wherein the differential connectivity [Δk^{˜}] for PGFC nodes with positive values shown in red indicate higher scaled connectivity in PSCN_{Caries} and negative values shown in blue indicate higher scaled connectivity in PSCN_{Caries-free}. The lower colored panel shown in FIG. 13A shows DCN clusters sorted by the number of PGFCs in cluster with the largest cluster being 0 and the smallest being 5. FIG. 13B shows a hive plot of taxonomic categories for DCN(Cluster-4), DCN(Cluster-2), and DCN(Cluster-5) with red and blue edges following the scheme in FIG. 13A. FIG. 13C shows the same hive plot shown in FIG. 13B but grouping the PGFCs by higher-level KEGG categories instead of taxonomic categories.

FIG. 14 illustrates Cardiobacterium hominis carbohydrate and ATP synthesis metabolism-centric DCN of PGFCs from DCN(Cluster-4), DCN(Cluster-2), and DCN(Cluster-5), according to some embodiments, that are grouped by either (1) taxonomy and higher-level KEGG categories for non-C. hominis PGFCs; and (2) C. hominis KEGG modules related to carbohydrate and ATP synthesis metabolism.

FIGS. 15A-C show PSCN connectivity with respect to functional categories according to some embodiments where FIG. 15A shows a connectivity profile for a caries PSCN with respect to KEGG categories, FIG. 15B shows a connectivity profile for a caries-free PSCN with respect to KEGG categories, and FIG. 15C shows a proportion of connectivity for KEGG modules with respect to a PSCN cluster.

FIGS. 16A and 16B show taxonomic and functional membership with respect to HCDCs according to some embodiments. FIG. 16A depicts a change in connectivity for each phyla with respect to HCDCs with connectivity enriched in caries (left) and connectivity enriched in caries-free (right). FIG. 16B depicts a change in connectivity for each KEGG category with respect to HCDCs with connectivity enriched in caries (left) and connectivity enriched in caries-free (right).

FIG. 17 shows a distribution of differential connectivities for the DCN shown in FIG. 10.

FIG. 18A illustrates a flow diagram of a mixed feature stacking classifier ensemble according to some embodiments, the stacking classifier ensemble including training data, base classifiers, meta classifier, and final prediction where X, y, and c/f represent the feature matrix, target vector, and base classifier respectively. Feature set specific base models feed into a meta classifier which outputs the final prediction.

FIG. 18B depicts a Venn diagram of PGFC features used to construct clf_{MCR} and clr_{CLR}.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

The headings provided herein are not limitations of the various aspects of the disclosure, which aspects can be understood by reference to the specification as a whole.

### Definitions

For purposes of the present disclosure, the following definitions are provided.

“Subject” as used herein, refers to a human or a non-human mammal including but not limited to a dog, cat, horse, donkey, mule, cow, domestic buffalo, camel, llama, alpaca, bison, yak, goat, sheep, pig, elk, deer, domestic antelope, or a non-human primate selected or identified for a diagnosis, treatment, inhibition, amelioration of a disease, disorder, condition, or symptom associated with or that may be associated with bacterial infection, bacterial colonization, bacterial growth, or dysbiosis, including but not limited to caries, precarious lesions (including “white spot” lesions), recurrent candidiasis, periodontal disease including periodontal inflammation and gingivitis, subgingival lesions including subgingival abscess, pulp infection, gingival infection with root involvement, cementum degradation, root abscess, gum rescission, or alveolar bone recession and/or degradation. As used herein, “subject” and “patient” may be used interchangeably.

As used herein a “sample” includes any sample obtained from a living system or subject, including fluid, blood, serum, tissue, or cells. In some embodiments, a sample is obtained by a swab of oral mucosae and/or tooth surfaces. Swabs may be taken from any or all of the soft palate; hard palate; surfaces of the tongue, throat, or tonsils. buccal, labial, or lingual gingiva: subgingival spaces including gingival pockets; lingual, buccal, labial, incisal, occlusal, or interdental surfaces of teeth. Samples may also comprise dentin, enamel, cementum, and/or alveolar bone, or may incorporate contents of the pulp cavity in either an inflamed or healthy state. In some embodiments, samples may comprise saliva and/or may be collected by rinse or lavage of any oral tissue or surface, whether naturally exposed or not. In some embodiments, samples may comprise nasal or lacrimal secretions. Samples can be in vivo or ex vivo.

As used herein, “prognosis” refers to the prospective determination of the likelihood of the occurrence of an event or condition, as well as an assessment of the likely severity of said event or condition. Such assessment of severity can include any clinical indicators, or predictors of morbidity, mortality, or other clinical outcome as would be known to one of ordinary skill in the art at the time the prognostic assessment was made. One of ordinary skill in the art will be presumed to be aware of the standard of care in the field at the time, as well as any foreseeable or obvious improvements to the standard of care, such as potential pharmaceutical, surgical, or other interventions which may ameliorate, exacerbate, or otherwise alter the course of the condition.

As used herein, “clinical diagnosis” refers to the diagnosis of a physiological condition made with reference to observations of a subject's physical or physiological state, including reference to such laboratory tests as may be included within the state of the relevant art at the time the diagnosis is made.

As used herein, “treatment” (and grammatical variations thereof such as “treat” or “treating”) refers to clinical intervention designed to alter the natural course of the individual or cell being treated during the course of clinical pathology. Desirable effects of treatment in some contexts include, but are not limited to, decreasing the rate of disease progression, amelioration or palliation of the disease state, and remission or improved prognosis.

As used herein, “prevention” includes providing prophylaxis with respect to occurrence or recurrence of a disease or the symptoms associated with a disease in an individual. An individual may be predisposed to, susceptible to, or at risk of developing a disease, but has not yet been diagnosed with the disease.

As used herein, an “effective amount” or “therapeutically effective amount” refers to an amount of therapeutic compound, such as a complement inhibitor, administered to a mammalian subject, either as a single dose or as part of a series of doses, which is effective to produce a desired therapeutic effect.

As used herein an “instruction” may include code in source code format, binary code format, executable code format, or any other suitable code format.

As used herein “sequencing module” includes devices that include, but are not limited to, standalone or add-on machines that sequence DNA, RNA, and/or polypeptides using any of the following technologies: next generation sequencing (NGS), nanopore sequencing (solid-state or biological nanopores), hifi sequencing, sequencing by binding, or sequencing by synthesis. Further, the sequencing module is directly and/or indirectly communicative with other electronic devices, including, but not limited to, desktop computers, laptops, servers, tablets, mobile devices, mobile phones, wearable devices, Universal Serial Bus (USB) flash drives in a variety of form factors, hard disk drives (HDD), solid state drives (SSD), USB and/or thunderbolt connectable drives (including, but not limited to, HDD and/or SSD), micro-flash cards, or any other equivalents thereof.

In some embodiments, the oral microbiome as referenced herein may comprise one or more bacteria of at least the following: Abiotrophia, Actinomyces, Aggregatibacter, Capnocytophaga, Cardiobacterium, Corynebacterium, Fusobacterium, Gemella, Granulicatella, Kingella, Lachnoanaerobaculum, Leptotrichia, Neisseria, Prevotella, Rodentibacter, Rothia, Streptococcus, Veillonella, or Weeksellaceae. Alternatively, the oral microbiome may comprise one or more bacteria from any combination of the following: Abiotrophia, Actinonyces, Aggregatibacter, Capnocytophaga, Cardiobacterium, Corynebacteriun, Fusobacterium, Gemella, Granulicatella, Kingella, Lachnoanaerobaculum, Leptotrichia, Neisseria, Prevotella, Rodentibacter, Rothia, Streptococcus, Veillonella, or Weeksellaceae. Alternatively, the oral microbiome may further comprise one or more nucleic acids from at least the following: Abiotrophia, Actinomyces, Aggregatibacter, Capnocytophaga, Cardiobacterium, Corynebacterium, Fusobacterium, Gemella, Granulicatella, Kingella, Lachnoanaerobaculum, Leptotrichia, Neisseria, Prevotella, Rodentibacter, Rothia, Streptococcus, Veillonella, or Weeksellaceae, or any combination thereof.

The oral microbiome may, in some embodiments, be delineated in terms of the presence of specific genes, gene clusters, expression units, operons, plasmids, metabolic clusters, or other genetically and/or metabolically determinable signifiers. For example, in some embodiments, the oral microbiome may include one or more genes within the metabolic families described in KEGG ID NO: M00001, M00002, M00003, M00005, M00006, M00007, M00011, M00016, M00018, M00021, M00022, M00023, M00048, M00050, M00051, M00052, M00082, M00083, M00093, M00125, M00140, M00149, M00157, M00159, M00165, M00167, M00169, M00307, M00345, M00346, M00529, M00549, M00579, M00632, M00846, M00866, and/or M00868 or any combination thereof.

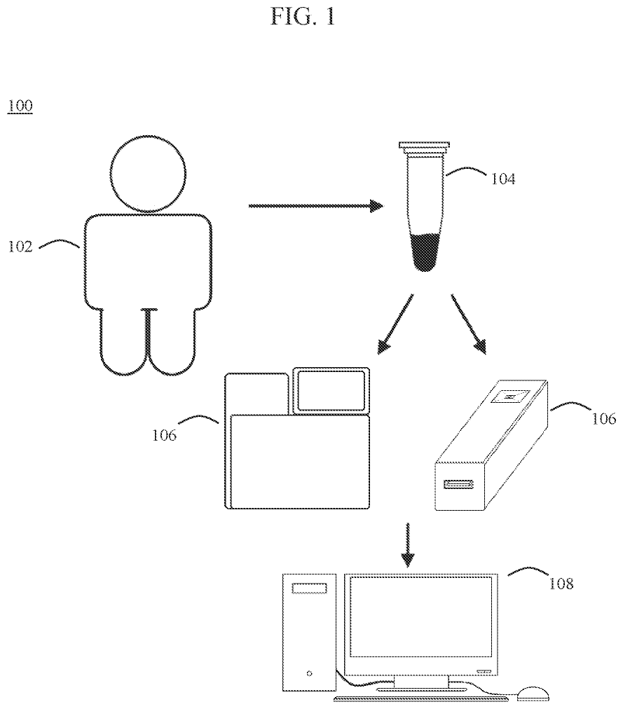

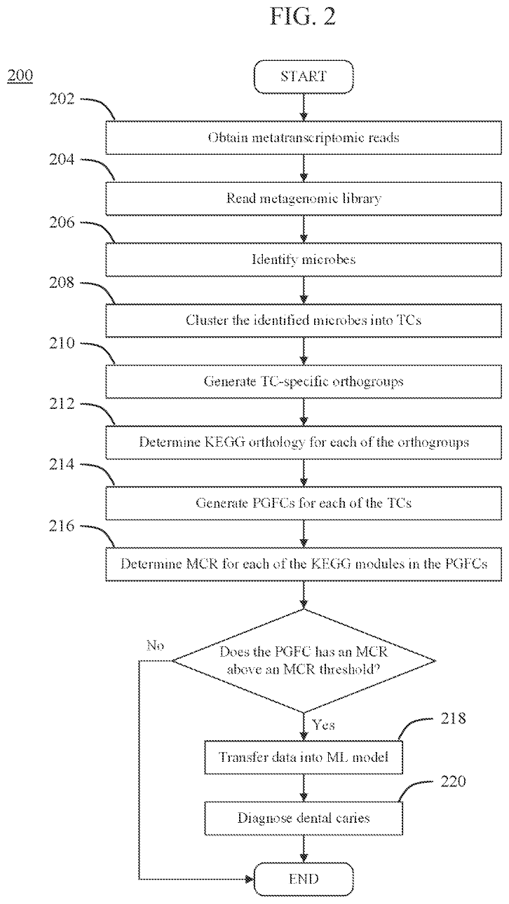

In some embodiments, a system described herein for predicting dental caries in a subject is provided with an illustrative example shown in FIG. 1. The system 100 may be used for the prediction of dental caries in a subject 102 or for providing a subject 102 with a dental care protocol or therapy. The system 100 comprises a sequencing module 106 that is configured to provide metatranscriptomic reads from an oral sample 104 from the subject 102. The oral sample 104 includes the oral microbiome of the subject. In some embodiments, the oral microbiome may include bacteria residing in any area, compartment, sub-compartment, lesion, recess, tissue, or surface of the oral cavity, including within the saliva, on or adjacent to lingual, buccal, or gingival tissues, within gingival pockets or recesses, within lingual crypts, or on or adjacent to the tissues of the throat or tonsils. Said oral microbiome may also include bacteria residing on, or attached to, any surface of a tooth, or any tissue thereof or associated ligament or bones, including but not limited to, enamel, dentin, cementum, alveolar bone, mandibular bone, within carious lesions, within a pulp cavity, palatine bone, maxillary bone and/or periodontal ligament.

In some embodiments, the oral sample 104 comprises supragingival plaque samples. In a non-limiting example, the supragingival plaque samples were obtained at the commencement of a dental examination. Prior to sample collection, subjects 102 were guided not to brush their teeth the night preceding the sample collection nor on the day of sample collection. Metadata were collected from three separate questionnaires completed by the parents during the period from consent to prior to the dental examination being undertaken. The clinical questionnaires consisted of a total of 132 questions to survey oral and medical health, dietary patterns, and development patterns, and dental hygiene. Visual inspection of the oral cavity followed International Caries Detection and Assessment System (ICDAS II). The ICDAS II was used to assess and define dental caries at the initial and early enamel lesion stages through to dentin and more advanced stages of the disease. Examiners were experienced clinicians who had undergone rigorous calibration and were routinely recalibrated across measurement sites to minimize error. Caries history in each subject 102 was initially reduced to a whole-mouth score and three classifications were utilized: no evidence of current or previous caries experience; evidence of current caries affecting the enamel layer only on one or more tooth surfaces; evidence of previous or current caries experience that has progressed through the enamel layer to involve the dentin on one or more tooth surfaces (including restorations or tooth extractions due to caries). For the purpose of phenotypic analysis, disease states from twins were classified as evidence of caries in enamel or dentin.

In some embodiments, as shown in FIG. 2 the system further comprises one or more processors in communication with the sequencing module; and a memory in communication with the one or more processors, the memory storing instructions that, when executed by the one or more processors, cause the one or more processors to at least: communicate with the sequencing module, such as sending instructions to transfer the metatranscriptomic reads from the sequencing module to the processor in block 202; and process instructions to read a metagenomic library from a database in block 204. In some embodiments, the metagenomic library may include reference oral microbiomes that may similarly include bacteria residing on, or attached to, any surface of a tooth, or any tissue thereof or associated ligament or bones, including but not limited to, enamel, dentin, cementum, alveolar bone, mandibular bone, within carious lesions, within a pulp cavity, palatine bone, maxillary bone and/or periodontal ligament. In some embodiments, the reference oral microbiomes may also include bacteria residing on, or attached to, any surface of a tooth, or any tissue thereof or associated ligament or bones, including but not limited to, enamel, dentin, cementum, alveolar bone, mandibular bone, within carious lesions, within a pulp cavity, palatine bone, maxillary bone and/or periodontal ligament. In some embodiments, the metagenomic library comprises a genome catalog for oral plaque obtained from a sex-balanced population.

In some embodiments, the processor further comprises a network module, which is configured to connect to a cloud-based server. In some embodiments, the system further comprises a network module, which is configured to connect to a cloud-based server. In some embodiments, the database is stored on the cloud-based server. In some embodiments, the sequencing module comprises a network unit, which is configured to connect to the cloud-based server. In some embodiments, the sequencing module is configured to communicate with the processor through the cloud-based server.

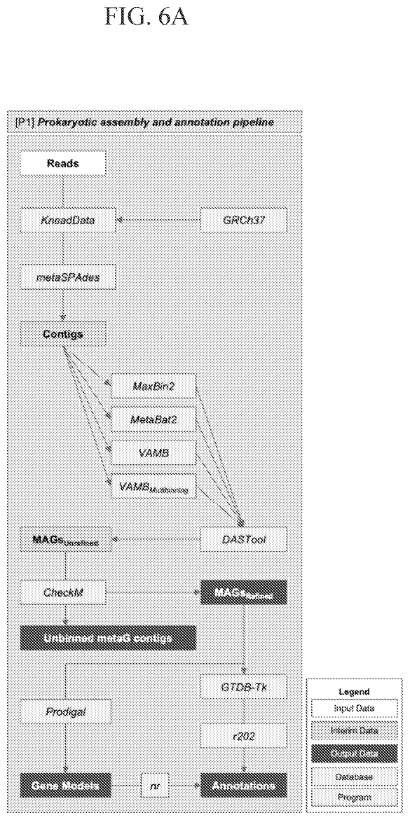

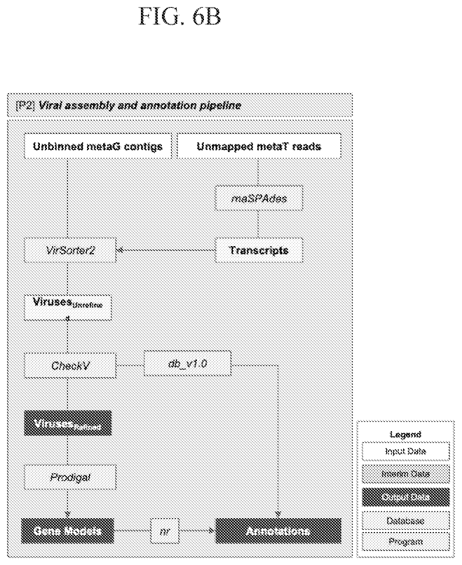

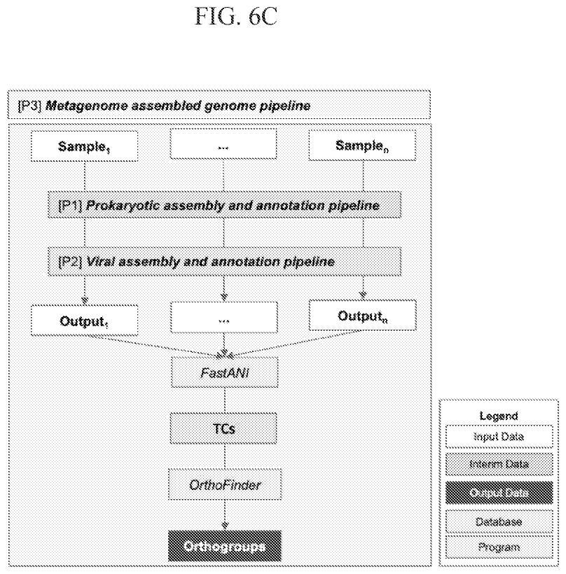

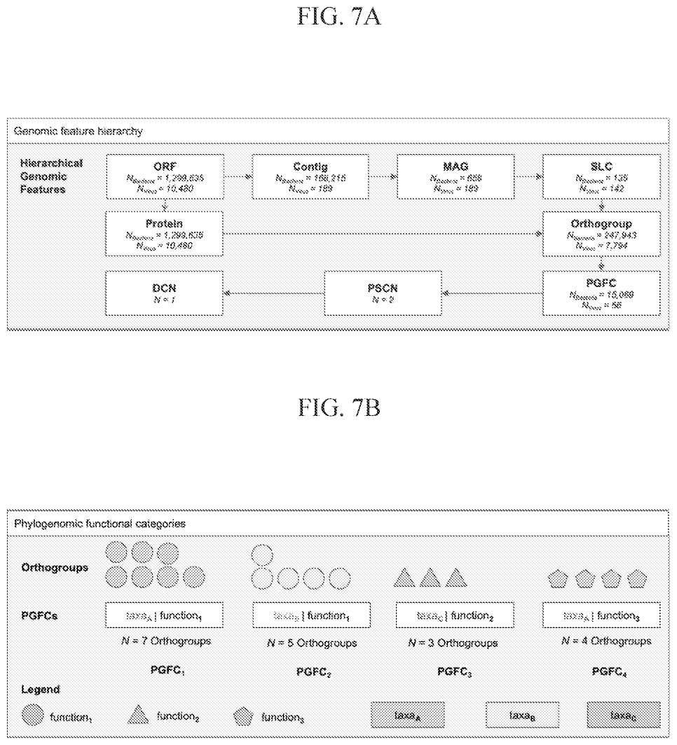

In some embodiments, the instructions, when executed by the one or more processors, may further cause the one or more processors to identify and cluster microbes identified in the oral sample into taxon clusters (TCs) in block 208 using the metatranscriptomic reads mapped to the metagenomic library as shown in FIGS. 2 and 6C. In some embodiments, the TCs are clustered based on ANI. In some embodiments, the ANI was determined by FastANI. In some embodiments, the AI was determined by MASH. In some embodiments, the ANI was determined by SourMash. In some embodiments, the ANI was determined by Dashing. In some embodiments, the ANI was determined by BBSketch. In some embodiments, a taxonomy of each of the TCs is assigned by a consensus of GTDB-tk classifications, Kraken classifications, and/or a consensus best-hit to existing protein databases. In some embodiments, the taxonomy of each of the TCs is assigned by GTDB-tk, MIDAS, Kraken 2, Kaiju, CAT BAT, Ganon, CCAletagen, and/or a consensus best-hit to existing protein databases. In some embodiments, these TCs include, but are not limited to, the species level clusters (SLCs) as shown in FIG. 7A. In a non-limiting example, an example dataset includes reads that include 1,310,118 ORFs to yield 255,737 SLC-specific orthogroups from 277 SLCs as shown in FIG. 7A, which would result in ˜32.7 billion non-redundant connections in a fully-connected co-expression network; an insurmountable dataset for exploratory analysis on most modern machines. Instead of using cumbersome filtering thresholds of orthogroups, a novel feature engineering technique was devised to allow seamless transitions from read↔ORF/orthogroup↔contig/MAG/SLC↔engineered feature using custom taxonomy fields and functional assignments (e.g., KEGG, MetaCyc, PFAM).

In some embodiments, the instructions, when executed by the one or more processors, may further cause the one or more processors to generate TC-specific orthogroups in block 208 shown in FIG. 2 for each of the TCs from protein clustering as shown in FIG. 7A. In some embodiments, the instructions, when executed by the one or more processors, may further cause the one or more processors to determine KEGG orthology in block 210 for each of the TC-specific orthogroups, or genes directly, using the metatranscriptomic reads mapped to the TC-specific orthogroups to provide KEGG modules for each of the TCs. In some embodiments, determining the KEGG orthology for each of the TC-specific orthogroups would allow generation of phylogenomic functional categories (PGFCs) in block 212 from a grouping of gene expression counts by the KEGG modules for each of the TCs. In some embodiments, the KEGG modules include, but are not limited to, KEGG ID NO: M00001, M00002, M00003, M00005, M00006, M00007, M00011, M00016, M00018, M00021, M00022, M00023, M00048, M00050, M00051, M00052, M00082, M00083, M00093, M00125, M00140, M00149, M00157, M00159, M00165, M00167, M00169, M00307, M00345, M00346, M00529, M00549, M00579, M00632, M00846, M00866, and/or M00868, or any combination thereof. Each of the above-referenced KEGG ID NO's are listed in Table 1. PGFCs are composite features that group metabolic functional information with genome-resolved taxonomy assignments as shown in FIG. 7B. In a non-limiting example, PGFCs are created in block 214 by grouping all of the TC orthogroups that had KEGG orthology, defined via KOFAMSCAN (a gene function annotation tool based on KEGG orthology and hidden Markov model), and extending the grouping up the hierarchy to modules with respect to taxonomy. Taxonomy for PGFCs were assigned to the FastAN cluster of origin. PGFCs were implemented using an EnsembleNetworkX v2021.06.24 Python package (38, 76) via the CategoricalEngineeredFeature class.

TABLE 1
List of taxon specific genes in the PGFCs

| KEGG ID | Name |
|---|---|
| M00001 | Glycolysis (Embden-Meyerhof pathway), glucose => pyruvate |
| M00002 | Glycolysis, core module involving three-carbon compounds |
| M00003 | Gluconeogenesis, oxaloacetate => fructose-6P |
| M00005 | PRPP biosynthesis, ribose 5P => PRPP |
| M00006 | Pentose phosphate pathway, oxidative phase, glucose 6P => ribulose 5P |
| M00007 | Pentose phosphate pathway, non-oxidative phase, fructose 6P == > ribose 5P |
| M00011 | Citrate cycle, second carbon oxidation, 2-oxoglutarate => oxaloacetate |
| M00016 | Lysine biosynthesis, succinyl-DAP pathway, aspartate => lysine |
| M00018 | Threonine biosynthesis, aspartate => homoserine => threonine |
| M00021 | Cysteine biosynthesis, serine => cysteine |
| M00022 | Shikimate pathway, phosphoenolpyruvate + erythrose-4P => chorismate |
| M00023 | Tryptophan biosynthesis, chorismate => tryptophan |
| M00048 | Inosine monophosphate biosynthesis, PRPP + glutamine => IMP |
| M00050 | Guanine ribonucleotide biosynthesis, IMP => GDP,GTP |
| M00051 | Uridine monophosphate biosynthesis, glutamine (+PRPP) => UMP |
| M00052 | Pyrimidine ribonucleotide biosynthesis, UMP => UDP/UTP, CDP/CTP |
| M00082 | Fatty acid biosynthesis, initiation |
| M00083 | Fatty acid biosynthesis, elongation |
| M00093 | Phosphatidylethanolamine (PE) biosynthesis, PA => PS => PE |
| M00125 | Riboflavin biosynthesis, plants and bacteria, GTP => riboflavin/FMN/FAD |
| M00140 | Cl-unit interconversion, prokaryotes |
| M00149 | Succinate dehydrogenase, prokaryotes |
| M00157 | F-type ATPase, prokaryotes and chloroplasts |
| M00159 | V-type ATPase, prokaryotes |
| M00165 | Reductive pentose phosphate cycle (Calvin cycle) |
| M00167 | Reductive pentose phosphate cycle, glyceraldehyde-3P => ribulose-5P |
| M00169 | CAM (Crassulacean acid metabolism), light |
| M00307 | Pyruvate oxidation, pyruvate => acetyl-CoA |
| M00345 | Formaldehyde assimilation, ribulose monophosphate pathway |
| M00346 | Formaldehyde assimilation, serine pathway |
| M00529 | Denitrification, nitrate => nitrogen |
| M00549 | Nucleotide sugar biosynthesis, glucose => UDP-glucose |
| M00579 | Phosphate acetyltransferase-acetate kinase pathway, acetyl-CoA => acetate |
| M00632 | Galactose degradation, Leloir pathway, galactose => alpha-D-glucose-IP |
| M00846 | Siroheme biosynthesis, glutamyl-IRNA => siroheme |
| M00866 | KDO2-lipid A biosynthesis, Raetz pathway, non-LpxL-LpxM type |
| M00868 | Heme biosynthesis, animals and fungi, glycine => heme |

In some embodiments, the instructions, when executed by the one or more processors, may further cause the one or more processors to determine a module completion ratio (MCR) in block 216 for each of the KEGG modules in the PGFCs. MCRs are calculated from the KEGG orthologs defined by KOFAMSCAN using MicrobeAnnotater v2.0.5 (77). In some embodiments, PGFCs are removed if they did not have MCR >50% in at least 20 samples, which, in one non-limiting example, results in 2,478 PGFCS representing 89 bacterial taxonomic units and 113 functional units from 8554 orthogroups. While not necessary to set the MCR threshold to be greater than 50%, the use of such a stringent threshold is preferable because many downstream associations may be misleading if only a small number of enzymes are represented in the module. In some embodiments, the instructions, when executed by the one or more processors, may further cause the one or more processors to retain the PGFCs having the MCR above the MCR threshold to obtain input data. In some embodiments, the MCR threshold for PGFCs that are retained is at least about 50%. In some embodiments, the MCR threshold for PGFCs that are retained is ≥50%. In some embodiments, the MCR threshold for PGFCs that are retained is >50%. Preferably, the MCR threshold for PGFCs that are retained is ≥50%. In a non-limiting example, a PGFC dataset includes 15,125 PGFCs incorporating 171 SLCs and 267 KEGG modules. While the dataset in this example is relatively dense, many of the KEGG modules in this example are incomplete with respect to a SLC in the sample. For increased biological interpretability, PGFCs with KEGG modules that were largely incomplete, MCR<50%, were filtered out to identify patterns with higher confidence amongst these high-level features; a feature that is not implemented in similar methodologies. From this filtering, the MCR-filtered PGFC dataset includes 2,478 PGFCs representing 89 taxonomic units and 113 functional units from 8,554 orthogroups; all of which are from bacterial SLCs. In the orthogroup-space, these features would amount to ˜37 million non-redundant connections but ˜3 million % in PGFC-space effectively compressing the information content by ˜92%, thereby making prototyping and data exploration practicable on modern computing machines.

A non-limiting example of genera weights related to MCR and CLR are shown in Tables 2A and 2B with respect to at least some of the genera found in the oral microbiome.

TABLE 2A
Genera weights

| | MCR Model | CLR Model |
|---|---|---|
| Species | Weights | Weights |
| Abiotrophia | 0.02169639 | 0.55227773 |
| Actinomyces | 0.02228207 | 0 |
| Aggregatibacter | 0.10660231 | 0 |
| Capnocytophaga | 0,13095846 | 1,74440758 |
| Cardiobacterium | 0.37312062 | 0.55515705 |
| Corynebacterium | 0 | 0.21299665 |
| Fusobacterium | 0,04749337 | 0.3910892 |
| Gemella | 0.3265982 | 0.59069761 |
| Granulicatella | 0.29359972 | 0.91492183 |
| Kingella | 0.3655833 | 0 |
| Lachnoanaerobaculum | 0.10621305 | 0 |
| Leptotrichia | 0.12892863 | 0.40556841 |
| Neisseria | 0.22391244 | 2.51212103 |
| Prevotella | 0 | 0.50488387 |
| Rodentibacter | 0.09055735 | 0 |
| Rothia | 0.10937885 | 1.16549935 |
| Streptococcus | 0.8232206 | 2.38965919 |
| Veillonella | 0.4778115 | 0.24073908 |
| Weeksellaceae | 0.07383617 | 0 |

TABLE 2B
Genera weights - MCR and CLR Model Weights summed

| | Model |
|---|---|
| | Weights |
| Species | (summed) |
| Abiotrophia | 0.57397412 |
| Actinomyces | 0.02228207 |
| Aggregatibacter | 0.10660231 |
| Capnocytophaga | 1.87536604 |
| Cardiobacterium | 0.92827767 |
| Corynebacterium | 0.21299665 |
| Fusobacterium | 0.43858256 |
| Gemella | 0.91729581 |
| Granulicatella | 1.20852154 |
| Kingella | 0.3655833 |
| Lachnoanaerobaculum | 0.10621305 |
| Leptotrichia | 0,53449704 |
| Neisseria | 2.73603347 |
| Prevotella | 0.50488387 |
| Rodentibacter | 0,09055735 |
| Rothia | 1.2748782 |
| Streptococcus | 3.21287979 |
| Veillonella | 0.71855058 |
| Weeksellaceae | 0.07383617 |

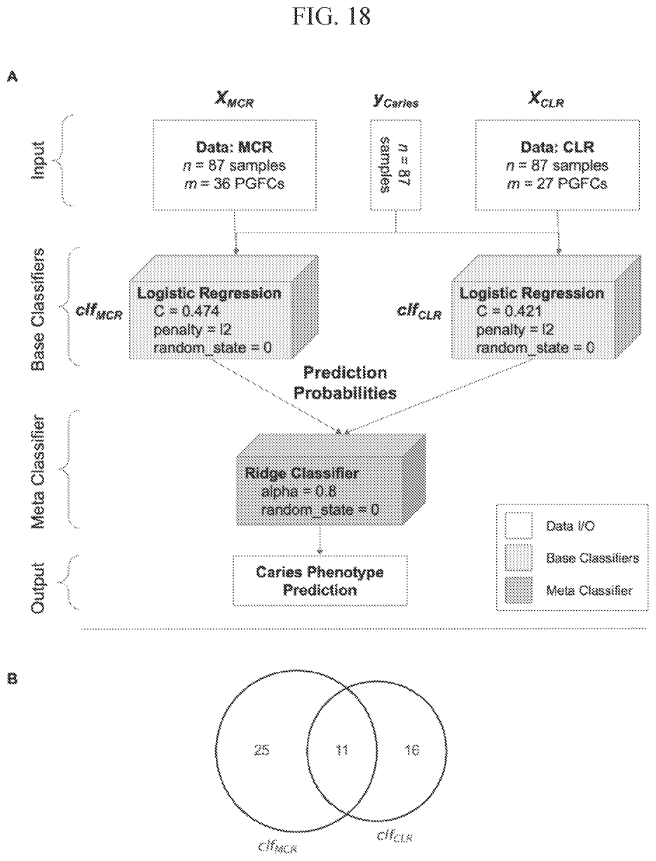

In some embodiments, the instructions, when executed by the one or more processors, may further cause the one or more processors to identify or predict, using a classifier model that includes variables selected by a feature selection machine learning algorithm, dental caries in said subject based on the input data. In some embodiments, the variables selected by the feature selection machine learning algorithm include, but are not limited to, expression of a species-specific metabolic pathway. Feature selection and predictive modeling was implemented to further evaluate PGFC features that were indicative of caries diagnosis. In particular, the Clairvoyance feature selection algorithm (37) was used to identify PGFCs that were able to accurately discriminate caries individuals from caries-free individuals. To allow for seamless interpretation with the network analysis, a set of PGFCs from a DCN (differential coexpression network) were used as input into the feature selection algorithm (see non-limiting example in Example 1), and this was implemented for PGFCs represented as MCR and as CLR transformed abundances to yield transformed abundances to yield two separate feature sets. The MCR is ≥50%, which indicated a greater completeness of the KEGG module(s) associated with each of the PGFCs than those PGFCs having an MCR<50%. When using the MCRs for each of the MCR-filtered PGFCs and the CLR feature sets as input into the feature selection machine learning algorithm in block 218, which also represented a novel type stacking ensemble, a dental caries vs. caries-free diagnosis was predicted in block 220, also as shown in FIGS. 18A-B. As shown in FIG. 18A, feature set specific base models feed into a meta classifier, which outputs the final prediction. In some embodiments, the dental caries vs. caries-fee diagnosis is at >99% accuracy.

The Clairvoyance feature selection algorithm is flexible and can use different algorithms that include, but are not limited to, linear regression, k-nearest neighbor, decision tree, neural networks, support vector machines, and Naïve Bayes along with more complex ensembles such as AdaBoost, Random Forest, Gradient Boosting Trees, and CatBoost. If any of the aforementioned algorithms are used, other feature selection algorithms can be applied including, but not limited to, recursive feature elimination, variance thresholding, false positive rate tests, false discovery rate tests, family-wise error rates, chi-squared tests, ANOVA tests, F-statistics, and/or mutual information tests.

In some embodiments, the instructions, when executed by the one or more processors, may further cause the one or more processors to generate a score based on an expression of genes from the metatranscriptomic reads as compared to genes within the KEGG module, and predicting or providing a diagnosis of dental caries in said subject when the score exceeds a threshold value. In some embodiments, when the diagnosis is for dental caries, the instructions, when executed by the one or more processors, may further cause the one or more processors to provide the subject a therapy for dental caries, such as removal of the dental caries, administration of dental fillings, providing a crown, root canal, or extraction. In some embodiments, the instructions further cause the one or more processors to output a dental care protocol or therapy for said subject, wherein the dental care protocol or therapy differs for subjects that have a score above the threshold value from subjects that have a score below the threshold value.

In some embodiments, the sequencing module comprises a sequencer having an interface for receiving nucleic acids obtained from said oral sample and possessing circuitry configured to sequence said nucleic acids. In some embodiments, the sequencer is a standalone machine as shown in FIG. 1. In some embodiments, the sequencer is a nanopore sequencer, the nanopore sequencer comprising a nanopore embedded in a membrane. In some embodiments, the nanopore sequencer is solid-state. In some embodiments, the nanopore sequencer is biological. In some embodiments, the nanopore sequencer is configured to be connectable to another electronic device via Thunderbolt, USB, eSATA, Bluetooth, NFC, RFID, wireless, or the like interfaces and/or protocols.

In some embodiments cDNA is generated from RNA obtained from said oral sample and said cDNA is sequenced or partially sequenced to yield the metatranscriptomic reads. In some embodiments, ribosomal RNA (rRNA) can be treated to remove rRNA before conversion to cDNA and sequencing library preparation, or after library preparation but prior to sequencing. Example treatments to remove rRNA include, but are not limited to, subtractive hybridization, exonucleolytic digestion of processed RNA with monophosphate at the 5′ end, duplex-specific nuclease-based digestion, electrophoretic size selection, sequence-specific blockage of reverse transcription, ribonuclease H (RNase H)-based selective digestion of rRNAs.

In some embodiments, the oral sample comprises dental plaque. In some embodiments, the oral sample comprises microbes such as bacteria, or virus, or nucleic acids from bacteria or virus. In some embodiments, the oral sample comprises Abiotrophia, Actinomyces, Aggregatibacter, Capnocytophaga, Cardiobacterium, Corynebacterium, Fusobacterium, Gemella, Granulicatella, Kingella, Lachnoanaerobaculum, Leptotrichia, Neisseria, Prevotella, Rodentibacter, Rothia, Streptococcus, or Weeksellaceae, or any combination thereof. In some embodiments, the oral sample comprises Neisseria and Bacteroidota or nucleic acids therefrom when the oral sample is from a subject lacking dental caries, such as a subject having a score that is less than the threshold value and/or, wherein the oral sample comprises Neisseria and Haemophilus_D parainfluenzae or nucleic acids therefrom when the oral sample is from a subject having dental caries, such as a subject having a score that is above the threshold value.

In some embodiments, the feature selection machine learning algorithm comprises differential co-expression analysis of the retained PGFCs. The differential co-expression analysis builds a differential co-expression network (DCN) from differences between phenotype-specific co-expression networks (PSCNs) using PGFCs. In some embodiments, DCNs reveal changes in connectivity between a reference (caries-free) and treatment (caries) network. As ensemble PSCNs are the building blocks of DCNs, the DCNs herein provide the same benefits with respect to outlier resistance. Previous approaches have used DCNs but did not use compositionally-aware association metrics or ensemble networks (21, 35). While differential abundance/expression analyses can be useful in identifying feature enrichment (e.g., OTU, MAG, ORF, gene, etc.), each method has their own caveats in assumptions about the data distributions (well characterized in (36) with the establishment of reference frames) and provide no information regarding differences in pairwise interactions; an essential perspective when studying diseases resulting from dysbiosis.

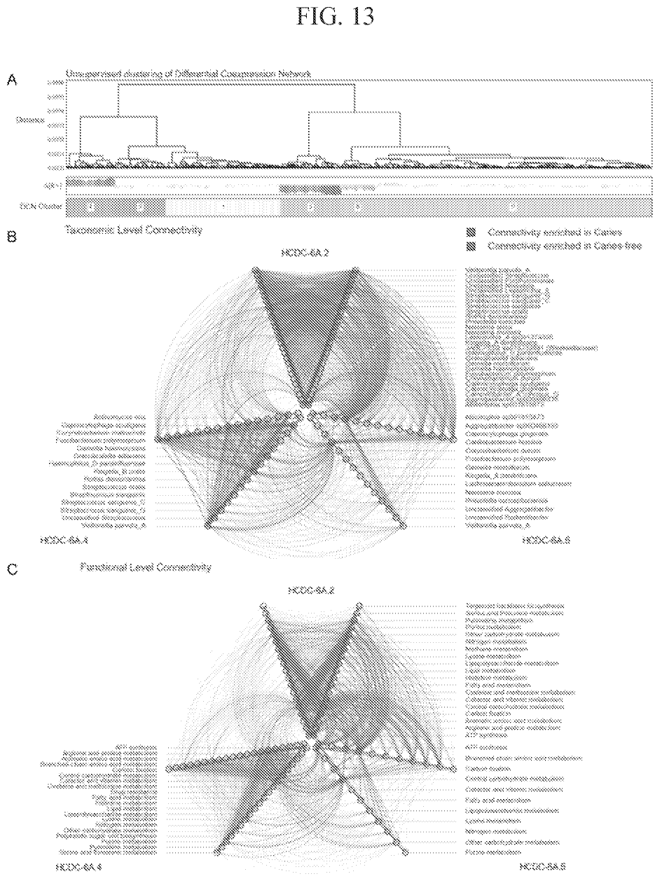

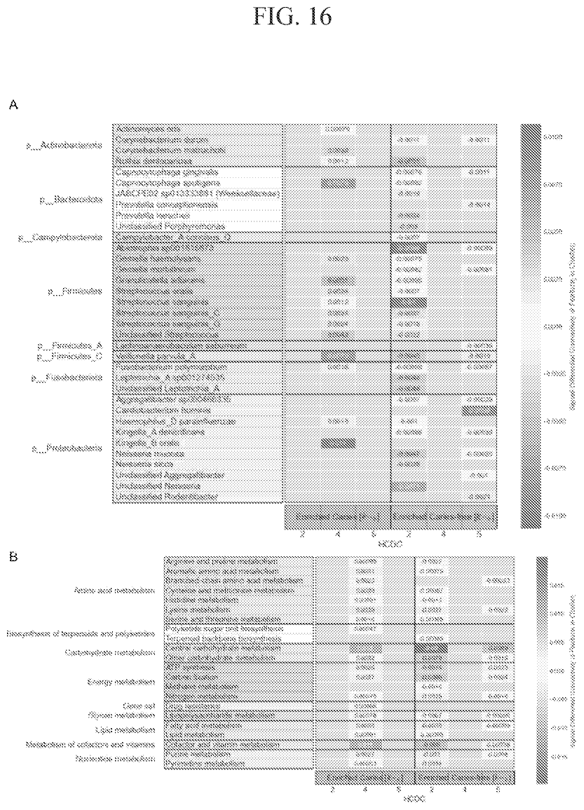

In a non-limiting example, using the PSCN_{Caries-free} as a reference network and PSCN_{Caries} as the treatment network, a DCN was constructed using 875 PGFCs from the community detection analysis for seamless cross-referencing between PSCNs and the DCN as shown in FIG. 13A-C. In the DCN, differential connectivity (denoted as Δk˜) is positive and negative when a connectivity is enriched in the caries and caries-free microbiomes, respectively. As shown, unsupervised clustering of the DCN revealed 6 clusters, of which there were 3 high connectivity DCN clusters (HCDC), each being diagnostic of phenotype. Also shown, HCDC-6A.4 had enriched connectivity in the caries microbiome while HCDCs-6A.2 and 5 had enriched connectivity in the caries-free microbiome. For the only HCDC with connectivity enriched in the caries microbiome (HCDC-6A.4), the differential connectivity was primarily from Capnocytophaga sputigena, Kingella_B oralis, Vellonella parvula_A, Streptococcus sanguinis, Streptococcus oralis, and unclassified Streptococcus (BC82, BC101, BC125) shown in FIG. 16A via carbohydrate and cofactor/vitamin metabolism shown in FIG. 16B.

In some embodiments, the feature selection machine learning algorithm further comprises hierarchical clustering and differential connectivity analysis to determine clusters of the PGFCs having a differential connectivity above a predictive threshold value for a phenotype, wherein, in some embodiments, the phenotype caries.

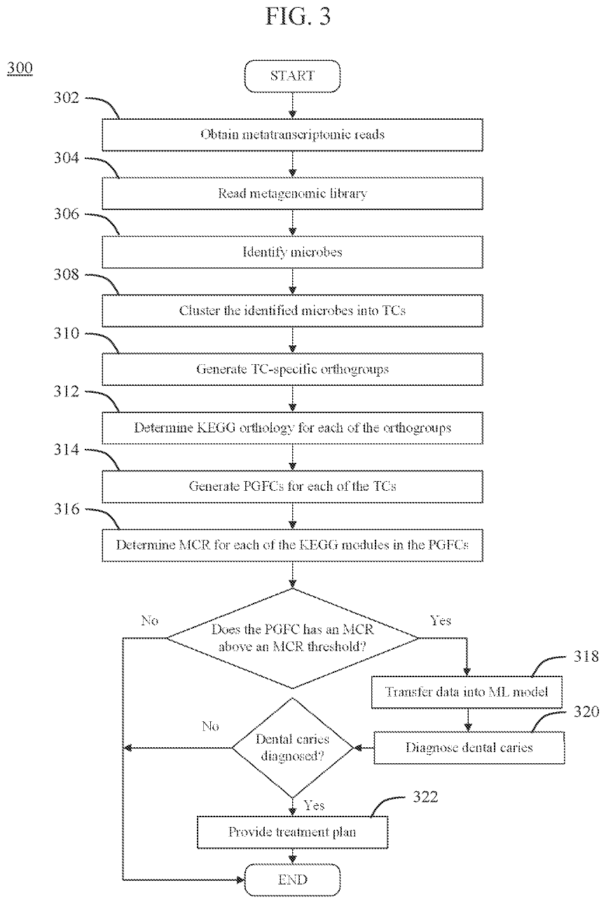

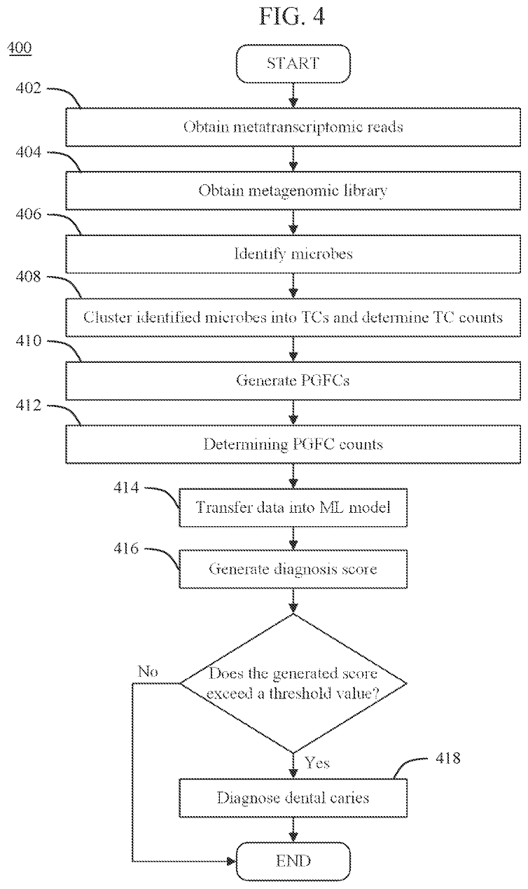

In another aspect of the invention, as shown in FIG. 3, a method 300 for predicting dental caries in a subject is provided, the method comprising: at a computer system including one or more processors, and memory storing one or more programs for execution by the one or more processors: obtaining, in an electronic format, metatranscriptomic reads in block 302 for an oral sample of a subject from a sequencing module, wherein the sequencing module comprises a sequencer having an interface for introduction of nucleic acids obtained from said oral sample. In some embodiments, the method further comprises, at the computer system including the one or more processors, the memory storing the one or more programs for execution by the one or more processors: obtaining, in an electronic format, a metagenomic library in block 304 from a database that is in communication with the computer system. In some embodiments, the method further comprises, at the computer system including the one or more processors, the memory storing the one or more programs for execution by the one or more processors: identifying microbes in block 306 that are actively expressing genes from the metatranscriptomic reads using the metagenomic library.

In some embodiments, the method further comprises, at the computer system including the one or more processors, the memory storing the one or more programs for execution by the one or more processors: clustering the identified microbes into TCs in block 308. In some embodiments, the method further comprises, at the computer system including the one or more processors, the memory storing the one or more programs for execution by the one or more processors: generating TC-specific orthogroups in block 310 for each of the TCs via protein clustering. In some embodiments, the method further comprises, at the computer system including the one or more processors, the memory storing the one or more programs for execution by the one or more processors: determining KEGG orthology in block 312 for each of the TC-specific orthogroups, or genes directly, from mapping metatranscriptomic reads to the TC-specific orthogroups to provide KEGG modules for each of the TCs. In some embodiments, the method further comprises, at the computer system including the one or more processors, the memory storing the one or more programs for execution by the one or more processors: generating PGFCs for each of the TCs in block 314 by grouping gene expression counts by the KEGG modules for each of the TCs. In some embodiments, the method further comprises, at the computer system including the one or more processors, the memory storing the one or more programs for execution by the one or more processors: determining an MCR for each of the KEGG modules in the PGFCs in block 316.

In some embodiments, the method further comprises, at the computer system including the one or more processors, the memory storing the one or more programs for execution by the one or more processors: retaining the PGFCs having the MCR above an MCR threshold to obtain input data in block 318. In some embodiments, the method further comprises, at the computer system including the one or more processors, the memory storing the one or more programs for execution by the one or more processors: providing the input data in block 318 into a feature selection machine learning algorithm configured to identify or predict dental caries in said subject from the input data, optionally, further generating a score based on an expression of genes from the metatranscriptomic reads as compared to genes within the KEGG module to predict or provide a diagnosis of dental caries in block 320 for said subject when the score exceeds a threshold value. In some embodiments, the method further comprises, at the computer system including the one or more processors, the memory storing the one or more programs for execution by the one or more processors: providing said subject a therapy for dental caries in block 322, such as removal of the dental caries, administration of dental fillings, providing a crown, root canal, or extraction.

In some embodiments, the method further comprises, at the computer system including the one or more processors, the memory storing the one or more programs for execution by the one or more processors: detecting orthogroups in the oral sample relative to a grouping of each of the retained PGFCs. In some embodiments, the method further comprises, at the computer system including the one or more processors, the memory storing the one or more programs for execution by the one or more processors: constructing phenotype-specific co-expression networks (PSCNs) by normalizing counts of the PGFCs with respect to a number of the detected orthogroups within the retained PGFCs.

In some embodiments, the method further comprises, at the computer system including the one or more processors, the memory storing the one or more programs for execution by the one or more processors: performing differential network analysis on the PSCNs to differentiate a caries expression state from a caries-free expression state and to generate differential co-expression networks (DCN) having a differential connectivity value. In some embodiments, the score comprises the differential connectivity value. In some embodiments, the threshold value is 0. In some embodiments, a dental caries microbiome is predicted when the differential connectivity value for the DCN is >0. In some embodiments, a dental caries-free microbiome is predicted when the differential connectivity value for the DCN is <0.

In some embodiments of the method, the one or more processors output a dental care protocol or therapy for said subject, wherein the dental care protocol or therapy differs for subjects that have a score above the threshold value from subjects that have a score below the threshold value.

In some embodiments of the method, cDNA is generated from RNA obtained from said oral sample and said cDNA is sequenced or partially sequenced to yield the metatranscriptomic reads. In some embodiments, rRNA can be treated to remove rRNA before conversion to cDNA and sequencing library preparation, or after library preparation but prior to sequencing. Example treatments to remove rRNA include, but are not limited to, subtractive hybridization, exonucleolytic digestion of processed RNA with monophosphate at the 5′ end, duplex-specific nuclease-based digestion, electrophoretic size selection, sequence-specific blockage of reverse transcription, ribonuclease H (RNase H)-based selective digestion of rRNAs.

In some embodiments of the method, the oral sample comprises dental plaque. In some embodiments of the method, the dental plaque comprises microbes such as bacteria, or virus, or nucleic acids from bacteria or virus. In some embodiments of the method, the oral sample comprises Abiotrophia, Actinomyces, Aggregatibacter, Capnocytophaga, Cardiobacterium, Corynebacterium, Fusobacterium, Gemella, Granulicatella, Weeksellaceae, Kingella, Lachnoanaerobaculum, Leptotrichia, Neisseria, Prevotella, Rothia, Rodentibacter, or Streptococcus, or any combination thereof. In some embodiments of the method, the oral sample comprises Neisseria and Bacteroidota or nucleic acids therefrom when the oral sample is from a subject lacking dental caries, such as a subject having a score that is less than the threshold value and/or, wherein the oral sample comprises Neisseria and Haemophilus_D parainfluenzae or nucleic acids therefrom when the oral sample is from a subject having dental caries, such as a subject having a score that is above the threshold value.

In some embodiments of the method, the computer system further comprises a network module, which is configured to connect to a cloud-based server. In some embodiments of the method, the database is stored on the cloud-based server. In some embodiments of the method, the sequencing module is configured to communicate with the computer system through the cloud-based server.

In some embodiments of the method, the TCs are clustered based on ANI. In some embodiments of the method, the ANI was determined by FastANI. In some embodiments, the ANI was determined by MASH. In some embodiments of the method, the ANI was determined by SourMash. In some embodiments of the method, the ANI was determined by Dashing. In some embodiments of the method, the ANI was determined by BBSketch. In some embodiments of the method, a taxonomy of each of the TCs is assigned by a consensus of GTDB-tk classifications, Kraken classifications, and/or a consensus best-hit to existing protein databases. In some embodiments of the method, the taxonomy of each of the TCs is assigned by GTDB-tk, MIDAS, Kraken 2, Kaiju, CAT/BAT, Ganon, CCMetagen, and/or a consensus best-hit to existing protein databases. In some embodiments of the method, the genomic library comprises a genome catalog for oral plaque obtained from a sex-balanced population.

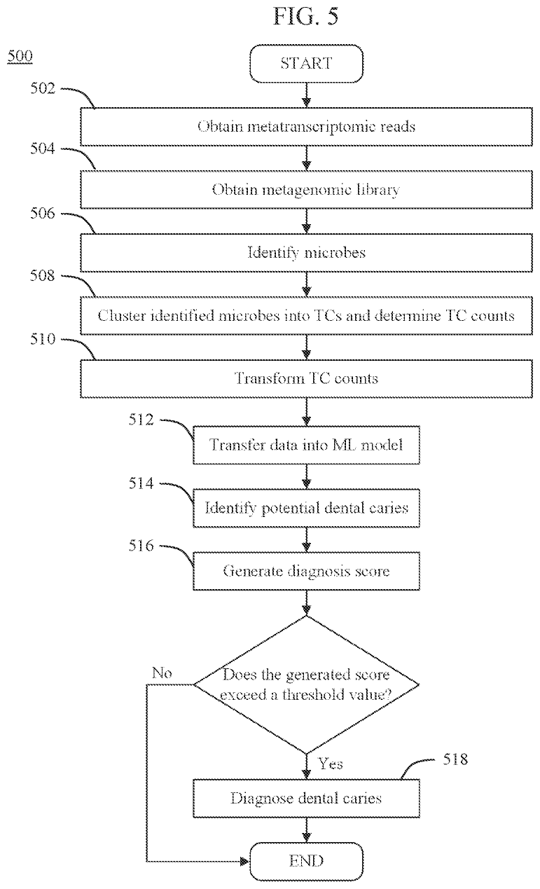

In another aspect of the invention, as shown in FIG. 4, a method 400 for predicting dental caries in a subject is provided, the method comprising: at a computer system including one or more processors, and memory storing one or more programs for execution by the one or more processors: obtaining, in electronic format, metatranscriptomic reads from a sequencing module in block 402, the sequencing module configured to sequence RNA molecules obtained from an oral sample provided by a subject. In some embodiments, the method further comprises, at the computer system including the one or more processors, the memory storing the one or more programs for execution by the one or more processors: obtaining, in electronic format, a metagenomic library in block 404 from a database that is communicative with the computer system. In some embodiments, the method further comprises, at the computer system including the one or more processors, the memory storing the one or more programs for execution by the one or more processors: identifying microbes in block 406 from the metatranscriptomic reads using the metagenomic library. In some embodiments, the method further comprises, at the computer system including the one or more processors, the memory storing the one or more programs for execution by the one or more processors: clustering the identified microbes into TCs and determining counts for the TCs in block 408. In some embodiments, the method further comprises, at the computer system including the one or more processors, the memory storing the one or more programs for execution by the one or more processors: clustering the metatranscriptomic reads into functional categories to generate PGFCs in block 410. In some embodiments, the method further comprises, at the computer system including the one or more processors, the memory storing the one or more programs for execution by the one or more processors: determining counts for the PGFCs in block 412. In some embodiments, the method further comprises, at the computer system including the one or more processors, the memory storing the one or more programs for execution by the one or more processors: feeding the counts of the TCs and PGFCs into a machine learning algorithm in block 414 that processes and sums the counts of the TCs and the PGFCs to generate a score in block 416, thereby predicting a diagnosis in block 418 of dental caries when the score exceeds a threshold value. In some embodiments of the method, the functional categories include KEGG modules.

In another aspect of the invention, as shown in FIG. 5, a method 500 for predicting dental caries in a subject is provided, the method comprising: at a computer system including one or more processors, and memory storing one or more programs for execution by the one or more processors: obtaining, in electronic format, metatranscriptomic reads in block 502 from a sequencing module, the sequencing module configured to sequence RNA molecules from an oral sample provided by a subject. In some embodiments, the method further comprises, at the computer system including the one or more processors, the memory storing the one or more programs for execution by the one or more processors: obtaining, in electronic format, a metagenomic library in block 504 from a database that is communicative with the computer system. In some embodiments, the method further comprises, at the computer system including the one or more processors, the memory storing the one or more programs for execution by the one or more processors: identifying microbes in block 506 from the metatranscriptomic reads using the metagenomic library. In some embodiments, the method further comprises, at the computer system including the one or more processors, the memory storing the one or more programs for execution by the one or more processors: clustering the identified microbes into TCs and determining counts for the TCs in block 508. In some embodiments, the method further comprises, at the computer system including the one or more processors, the memory storing the one or more programs for execution by the one or more processors. transforming the TC counts in block 510 using center log-ratio (CLR) into input data. In some embodiments, the method further comprises, at the computer system including the one or more processors, the memory storing the one or more programs for execution by the one or more processors: feeding the input data into a feature selection machine learning algorithm in block 512 to identify dental caries from the input data in block 514, and optionally, generating a score in block 516, thereby predicting a diagnosis of dental caries in block 518 when the score exceeds a threshold value.

Various aspects of the disclosure will now be described with regard to certain examples and embodiments, which are intended to illustrate but not limit the disclosure. Although the examples and embodiments described herein will focus, for the purpose of illustration, one specific calculations and algorithms, one of skill in the art will appreciate the examples are to illustrate only, and are not intended to be limiting. For example, although some examples are presented in the context of systems and methods for predicting the oral status of a subject (particularly, but not limited to, caries), the disclosed systems and methods can be configured or utilized to achieve the disclosed results in other implementations. Further, although certain examples are presented as achieving benefits for predicting the oral status of the subject, it will be appreciated that the disclosed examples of the systems and methods yield high predictability with regards to predicting caries status of a subject and provide additional treatment plans and options to the subject and the health care worker.

### Example 1

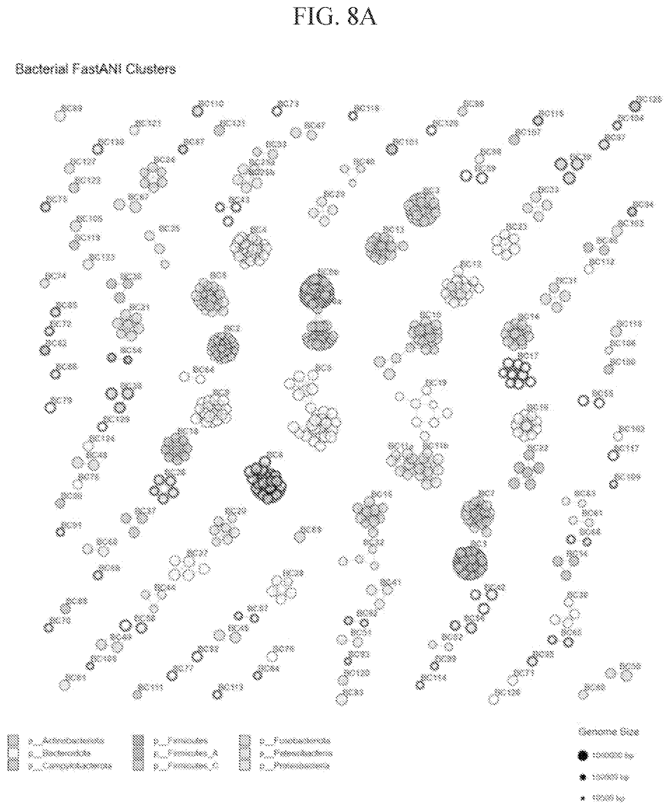

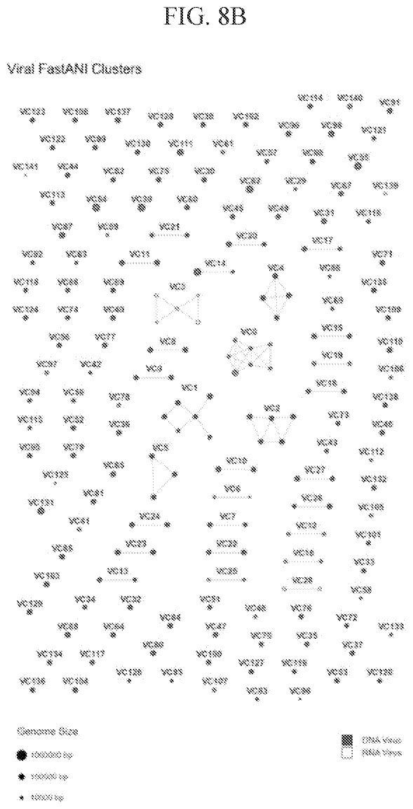

A supragingival dental plaque microbiome of 87 children was characterized (demographics shown in Table 3), which generated 658 bacterial MAGs and 189 viral MAGs (179 DNA viruses and 10 RNA viruses) as shown in FIGS. 8A-B with transcriptional profiling and gene-expression network analysis. The bacterial MAGs clustered (95% Average Nucleotide Identity (ANI)) into 135 species-level clusters (SLC) that represented 49 hitherto unclassified species, where 26 of which were classified as Pastescibacteria candidate phyla radiation (CPR; 6 Gracilibacteria/SRI, 43 Sacharbacteria) and a total of 69 CPR MAGs collectively. Of the non-CPR SLCs, 31 Bacteroidota, 22 Proteobacteria, 21 Actinobacteriota, 23 Firmicutes (A/C), 8 Fusobacteriota, and 4 Campylobacteriota, which are shown in Table 4. The DNA and RNA viruses were clustered into 137 and 5 unique SLCs, respectively. Most of the DNA viruses were classified as Caudovirales, of unknown species, associated with the human oral (42 SLCs), gut (41 SLCs including 1 Inoviridae), human respiratory (1 SLC), and non-specific environments (1 SLC). Aside from these unknown species, several Caudovirales phages for Arthrobacter (7 SLCs), Streptococcus (4 SLCs), Klebsiella, Haemophilus, Pasteurella, Pseudomonas, and Burkholderia were identified. Other than Caudovirales, Streptococcus satellite phages (2 SLCs), unclassified CRESS-DNA Parvovirus associated with the human gut (2 SLCs), and an unclassified virus associated with the human oral environment were identified. Most of the RNA viruses were Escherichia phages (4 SLCs) designated as Qbeta BZ1, MS2, and BZ13 strains but a novel virus with no close taxonomic classification was uncovered. The bacterial and viral SLCs contained 64 and 113 singleton clusters; individual genomes that did not share 95% ANT with any other organisms in the dataset. No archaea, eukaryote, or novel bacterial genera beyond the CPR were detected.

TABLE 3
Cohort Sample Metadata. Overview of sample size for cohort
with respect to phenotype and several metadata.

| Metadata | Caries | Caries-free |
|---|---|---|
| Age (μ, σ², min, max) | (8.09, 2.7, | (7.82, 2.21, |
| | 5.5, 10.9) | 5.4, 10.8) |
| Sex (Female) | 19 | 31 |
| Sex (Male) | 17 | 20 |
| Center (CBRG) | 21 | 28 |
| Center (MCRI) | 15 | 23 |
| Total Samples | 36 | 51 |

TABLE 4
Bacterial SLCs and MAGs with respect to phylum. The number of
bacterial MAGs, SLCs, and novel species with respect to phyla.

| | | | Novel Species |
|---|---|---|---|
| Bacterial Pbyla | SLCs | MAGS | (SLCs) |
| p_{——}Actinobacteriota | 21 | 153 | 3 |
| p_{——}Bacteroidota | 31 | 160 | 13 |
| p_{——}Campylobacterota | 4 | 4 | 3 |
| p_{——}Firmicutes | 16 | 35 | 4 |
| p_{——}Firmicutes A | 4 | 6 | 2 |
| p_{——}Firmicutes C | 3 | 62 | 0 |
| p_{——}Fusobacteriota | 8 | 40 | 1 |
| p_{——}Patescibacteria | 26 | 69 | 18 |
| p_{——}Proteobacteria | 22 | 129 | 5 |
| Total | 135 | 658 | 49 |

The metapantranscriptomics approach utilized in this example collapsed 1,248,783 orthogroups (ORFs) into 255,737 SLC-specific orthogroups (247,943 bacterial and 7,794 viral), thereby reducing the dimensionality by 80% with minimal loss in information content. Without being bound by theory, by using SLC-specific orthogroups, a “bag-of-genomes” paradigm was maintained, as opposed to that of a “bag-of-genes,” which preserves natural hierarchical structures inherent in ecology.

There was only one taxonomic database discrepancy that was observed, where M-1507-144.A_MAXBIN2 bin.008 was classified by GTDB-Tkas a novel Tannerella but had this MAG clustered in BC14 with 10 Peptidiphaga sp000466175 with high confidence (>95% ANI via FastAN) which suggests an update to the GCF_003033925.1 reference taxonomy in NCBI. This result is further strengthened by the CheckM basal classification of the Actinobacteria phylum.

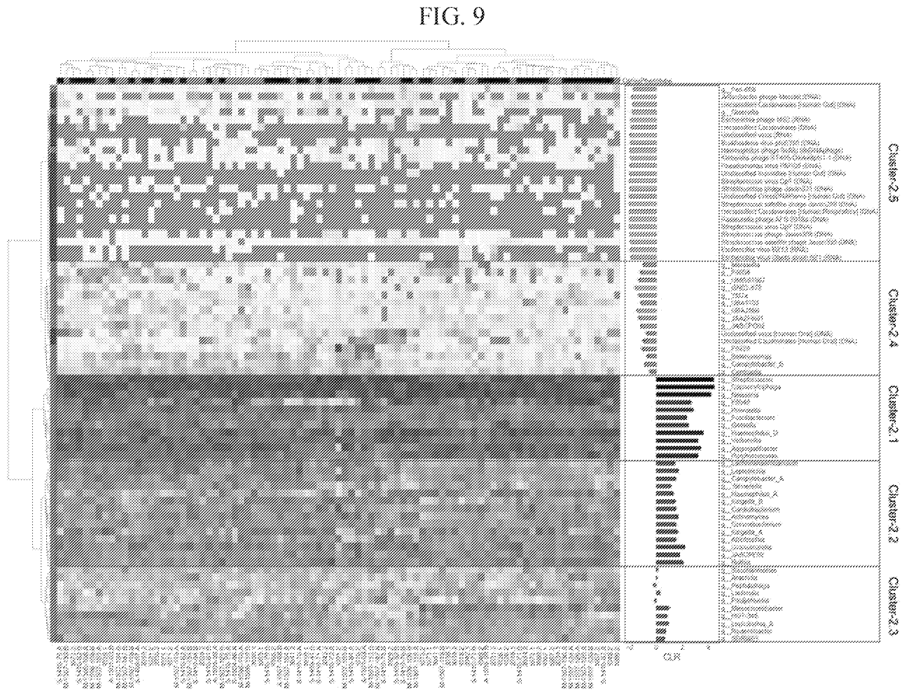

Relative taxonomic expression and abundance. FIG. 9 shows clustering using genome-resolved gene expression (specifically Euclidean distance and Ward linkage) that group subjects into 5 distinct clusters, but these expression patterns were not able to discriminate samples based on the presence or absence of caries. CLR-transformed abundances of taxa from metatranscriptomics are shown in FIG. 9 where row colors represent bacterial (teal) or viral (maroon) MAGs while the column colors represent caries (red) or caries-free (black) phenotpes. The silhouette scores using Aitchison distance were measured against caries status and observed average scores close to zero (|x̅ₛᵢₗₕₒᵤₑₜₜₑ|<0.003) for bacterial and viral microbiomes in both metatranscriptomics and metagenomics datasets indicating minimal phenotype partitioning capacity using individual features.

As these expression patterns are in CLR, values close to 0 can be considered basal community-level expression and close to the geometric mean of expression values for the microbiome. A core bacterial supragingival plaque microbiome at the genus level is shown, as almost every genus is transcriptionally active in every sample (Clusters 2.1-2.4), regardless of phenotype, but this is not the case for either DNA or RNA viruses. Most of the viruses were detected with low expression and grouped in Cluster-2.5. In Cluster-2.5, there are a few DNA viruses that are detected in almost every sample including Streptococcus satellite phage Jaamn335, unclassified Caudovirales associated with human gut, Burkholderia phage phiE255, Haemophilus phage SuMu, and Klebsiella phage ST405-OXA48phiJ with Escherichia phage MS2 as the only core RNA virus. Cluster-2.4 contains an unclassified human oral DNA virus and an unclassified oral Caudovirales that are transcriptionally active in every sample at modest levels on par with many bacteria in the microbiome. Cluster-2.1, the one with highest overall transcript abundance, genera from Bacteroidota, Proteobacteria, Firmicutes(C), and Fusobacteriota was mostly observed. The most transcriptionally active genera in Cluster-2.1 are Capnocytophaga, Streptococcus, Neisseria, Haemophilus_D, Aggregibacter, Porphyromonas, and Veillonella with modest expression from Prevotella, Fusobacterium, Gemella, and Alloprevotella (i.e., F0040). Cluster-2.2, with CLRs of 1-2, includes genera that appear in downstream analysis including Cardiobacterium, Corynebacterium, and Tannerella. Cluster-2.3 is the cluster with baseline transcriptional activity (i.e., CLR close to 0) which contains all the Saccharibacteria and SRI CPR clade.

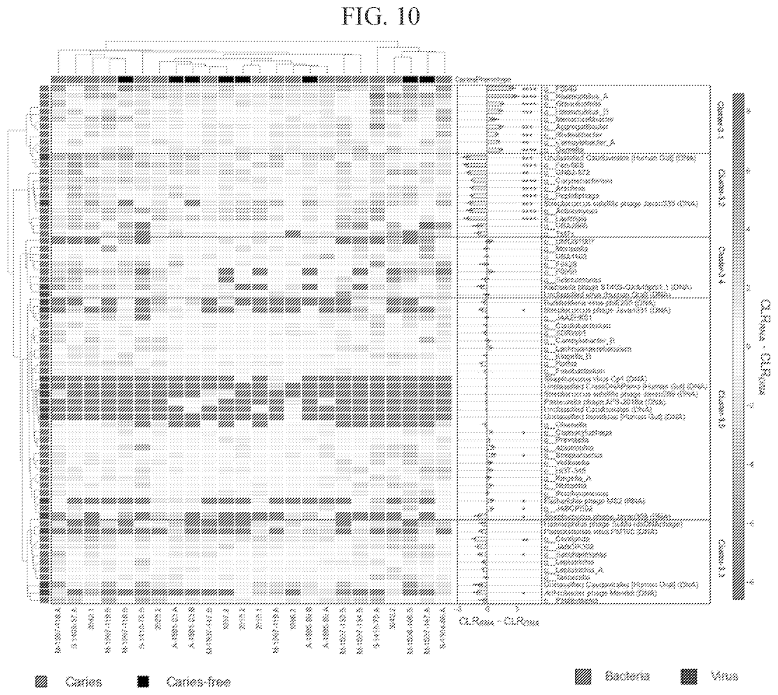

RNA:DNA ratios from the 26 overlapping metatranscriptomic and metagenomic samples were also investigated as shown in FIG. 10. Based on clustering of RNA:DNA ratios, 5 distinct groupings were observed (Clusters-3.1-5). The most prominent findings are from Clusters-3.1, 3.2 where taxa have highest and lowest RNA:DNA ratios, respectively. Cluster-3.1, the most transcriptionally active, included Haemophilus A and Alloprevotella, the most active genera, as well as Aggregibacter, Gemella, and Campylobacter_A. Cluster-3.2 has a more uniform distribution of low RNA:DNA ratios and contained Gracilibacteria, Saccharibacteria, Streptococcus satellite phage.Javan335, and an unclassified Caudovirales phage associated with the human gut. Clustering of these RNA:DNA ratios also did not differentiate subjects based on phenotype.

In terms of alpha diversity, any difference in bacterial richness for metatranscriptomics (\tilde{x}=131 SLCs) or metagenomics (\tilde{x}=134 SLCs) datasets between caries and caries-free microbiomes were not observed (Mann-Whitney p>>0.05). For viral richness, difference in the metatranscriptomics dataset (\tilde{x}=26 SLC) was not observed, however a slight enrichment in viral richness in the caries microbiome was observed (\tilde{x}_{Caries}=34.5 SLCs, \tilde{x}_{Caries-free}=30 SLCs; Mann-Whitney p=0.026).

Differential expression analysis was performed between caries and caries-free cohorts at the taxonomic level (SLC expression) and PGFC level (engineered taxonomy-functional composite features). Statistically significant components were not observed, neither SLCs nor PGFCs, using compositionally-aware methods such as ANCOM and ALDEx2. However, the lack of clear taxonomic or functional differences between the cohorts suggests interactions between variables is important, illustrating the need of differential networks to interrogate the caries and caries-free microbial systems.

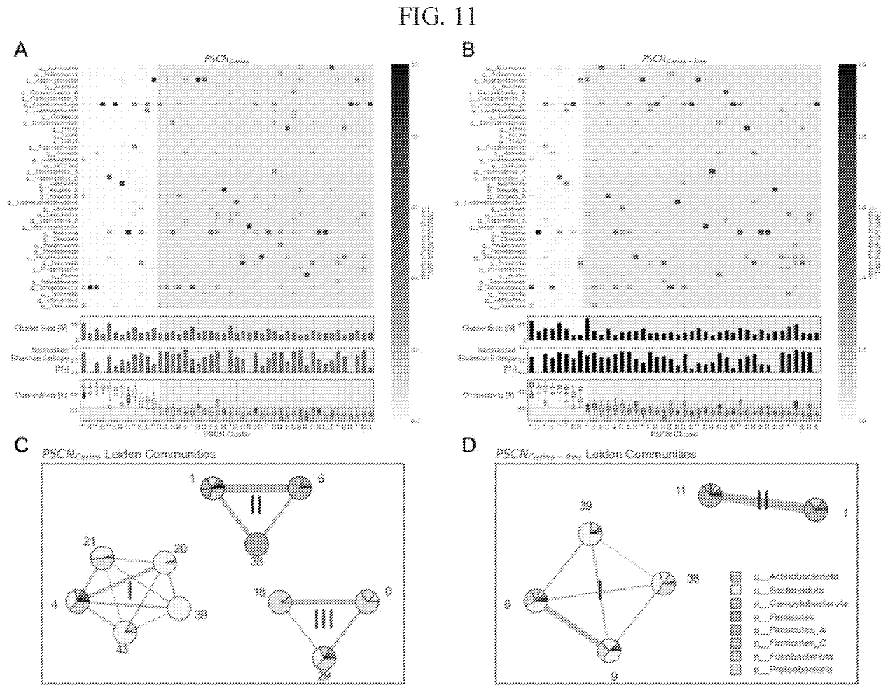

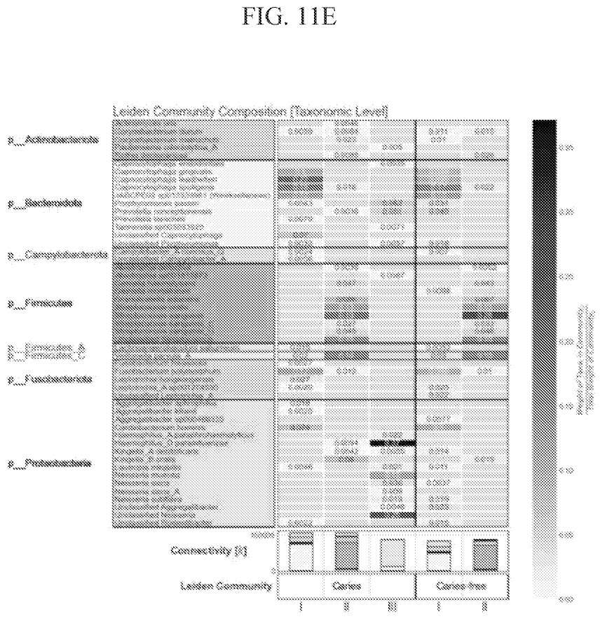

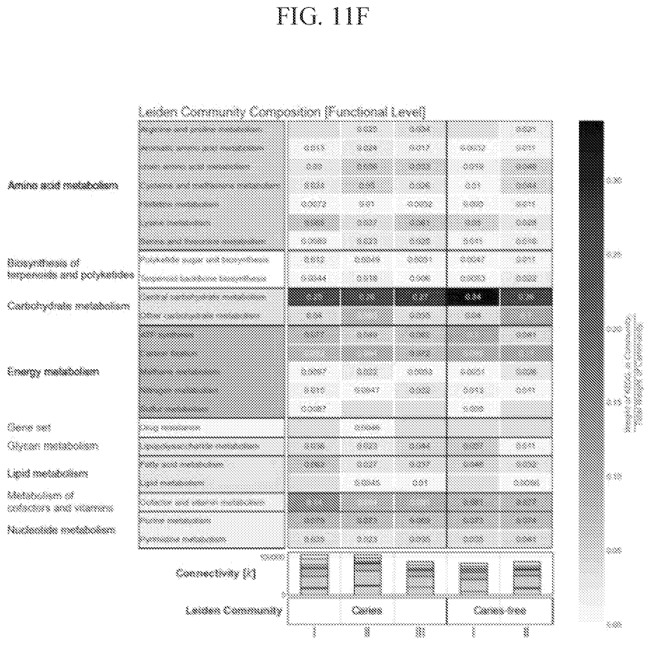

Phenotype-specific co-expression networks (PSCNs) reveal unique taxonomic and metabolic characteristics. As the caries phenotype is a multifactorial disease (7), a natural approach for investigating associations would be through network analysis as such methodologies are useful for modeling complex systems with unknown structure. To be specific, the true structure (if one exists) of microbial interactions within each phenotype in the dataset is unknown a priori. Therefore, the structure of each network using data-driven approaches must be inferred. Using an ensemble approach, compositionally-aware co-expression networks were computed, with PGFCs, an engineered feature based on taxonomy and functional potential (see Methods), as nodes (N_{nodes}=2,478) and rho proportionalities as edge weights (N_{edges}=3,069,003), for caries and caries-free microbiomes (PSCN_{Caries} and PSCN_{Caries-free}, respectively). The total connectivity of the PSCN_{Caries} was 301,163.9 k with PSCN_{Caries-free} ˜7% lower (279,832.1 k). Unsupervised clustering of the PSCNs sorted by median connectivity revealed clusters heterogeneous with respect to taxonomy, and a sharp drop off in connectivity at 250 k (FIG. 11A-B). In this high connectivity range, there are 12 PSCN_{Caries} clusters (749 PGFCs) and 8 PSCN_{Caries-free} clusters (555 PGFCs) which will be referred to as high connectivity PSCN clusters (HCPC).

One approach in computing homogeneity is via normalized entropy and, in this context, can be interpreted as cluster homogeneity where low entropy translates to a cluster being dominated by a single taxa (more homogenous) and high entropy as taxa being evenly distributed within a cluster (more heterogeneous). The most highly connected cluster in both PSCNs is Cluster-1 (HCPC-4A.1 and HCPC-4B.1), which is the second largest cluster in each network and one of the most heterogenous with respect to taxonomy. A modest trend that HCPCs in the caries microbiome have higher taxonomic homogeneity than the caries-free microbiome was seen. The caries HCPCs tend to have lower normalized entropy than the caries-free HCPCs especially compared to when considering all clusters; though, the number of observations was not sufficient for this statistical analysis and these results will not be further explored.

Despite the caries microbiome HCPCs being slightly more homogenous, the highest connectivity PSCN_{Caries} cluster (HCPC-4A.1) is one of the most heterogenous clusters in the system. The majority of HCPC-4A.1 connectivity (82%) is from Veillonella (30.2%), Streptococcus (18.1%), Granulicatella (17.4%), and Kingella_B (16.2%). The remaining caries HCPCs are enriched in Streptococcus, Capnocytophaga, Haemophilus D, JABCPE02 (f_Weeksellaceae), Neisseria, Cardiobacterium, and Aggregatibacter. The highest connectivity cluster in PSCN_{Caries-free} is HCPC-4B.1 whose connectivity is primarily from Streptococcus (63.6%; 54.8% from S. sanguinis), Veillonella parvula A (22.2%), and Granulicatella adiacens (10.4%). The remaining caries-free HCPCs are enriched in Neisseria, Capnocytophaga, Fusobacterium, Haemophilus D, JABCPE02 (f Weeksellaceae), and Prevotella. A substantial overlap in high connectivity genera but the cluster membership of these genera is phenotype-specific and these configurations may provide key insight into how a system, whether caries or caries-free, stabilizes.

The second highest connectivity HCPCs in both caries (HCPC-4A.32) and caries-free (HCPC-4B.22) microbiomes are homogenous for Streptococcus and Neisseria, respectively. Connectivity from HCPC-4A.32 is mainly derived from S. sanguinis (98%) and a novel Streptococcus species (BC101, 2%) while connectivity from HCPC-4B.22 is 100% attributable to a novel Neisseria species (BC6). Additionally, HCPC-4A.39 as another homogenous caries HCPC for Capnocytophaga sputigena was observed. In both PSCNs, carbohydrate metabolism (27% PSCN_{Caries}, 36% PSCN_{Caries-free}) and cofactor/vitamin biosynthesis (10.7% PSCN_{Caries}, 8.2% PSCN_{Caries-free}) are attributable to most of the connectivity (FIGS. 7A-B). Glycolysis, gluconeogenesis, and pentose phosphate are heterogenous amongst the HCPCs regardless of phenotype. The citric acid cycle was responsible for the majority of the carbohydrate connectivity in PSCN_{Caries} HCPC-4A.29; a heterogenous cluster enriched in Neisseria and Prevotella. Several cofactor and vitamin metabolic pathways were common amongst the HCPCs. In particular, C1-unit interconversion (M00140), tetrahydrofolate biosynthesis (M00126), and heme biosynthesis (M00121) were the most common.

Community detection algorithms such as Louvain (29) and, its updated successor, Leiden (30) have been used to investigate the structure of large and complex networks. The former has been used to study various biological networks (31-34) while Leiden is new and sparingly applied to biological systems, it addresses defects associated with Louvain (30). As these algorithms are stochastic, an iterative version of the Leiden community detection algorithm was used to investigate how these phenotype-specific HCPCs are structured and how the HCPCs partition into tightly connected high-confidence communities in an induced graph. The caries HCPCs naturally partition into Communities-4C.I-III while the caries-free HCPCs partition into Communities-4D.I-II (FIGS. 11C-D). Leiden communities reveal similar coexpression of two complementary configurations in both PSCNs: 1) majority Bacteroidota (PSCN_{Caries} Community-4C.I and PSCN_{Caries-free} Community-4D.I); and 2) majority Firmicutes via Streptococcus (PSCN_{Caries} Community-4C.II and PSCN_{Caries-free} Community-4D.II) (FIGS. 11E-F).

Community-4C.I and 4D.I have high overlap in taxonomic membership, but they also have several unique taxa that may provide insight into phenotype-specific system states. Interestingly, no Neisseria were observed in PSCN_{Caries} Community-4C.I but high Neisseria genus-level membership was observed in the complementary PSCN_{Caries-free} Community-4D.I (FIG. 11E). However, in PSCN_{Caries}, high Neisseria genus-level membership in Community-4C.III coexpressed with more Neisseria and Haemophilus was observed (FIG. 11E). Neisseria are highly connected in both PSCNs but their community membership, the taxa they are interacting with, is phenotype specific. More specifically, Neisseria appears to shift from high coexpression with Bacteroidota (Capnocytophaga, Porphyromonas, Prevotella conceptionensis, and JABCPE02 (f_Weeksellaceae)) in the caries-free cohort to other Neisseria and Haemophilus (Haemophilus_A paraphrohaemolyticus and Haemophilus_D parainfluenzae) in the caries cohort. The connectivity of Haemophilus_D parainfluenzae and an unclassified Neisseria (BC6) is relatively high in PSCN_{Caries} Community-4C.III and these taxa are completely absent from the Neisseria enriched community in PSCN_{Caries-free}.

In terms of metabolism, drug resistance is only observed in PSCN_{Caries} Community 4C.II, specifically Streptococcus sanguinis beta-Lactam resistance. Both caries and caries-free microbiomes lack arginine, proline, and lipid metabolism in the Bacteroidota-centric communities (PSCN_{Caries} Community-4C.I and PSCN_{Caries-free} Community-4D.1) but provide these pathways in the Firmicutes-centric communities (PSCN_{Caries} Community-4C.II and PSCN_{Caries-free} Community-4D.II). Conversely, these Firmicutes-centric communities lack sulfur metabolism which appears to be provided by Bacteroidota-centric community. Central carbohydrate metabolism connectivity is much higher in the Bacteroidota-centric PSCN_{Caries-free} Community-4D.I relative to all of the other communities which may suggest that the taxa and central carbohydrate mechanisms in this community promote a healthy oral microbiome.

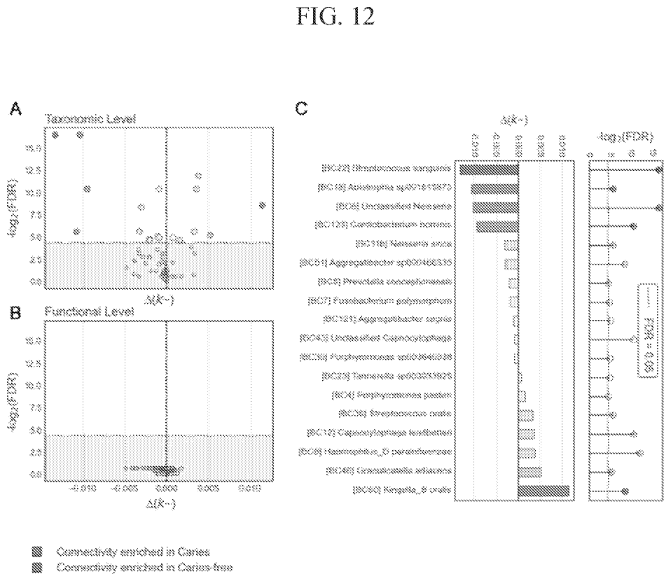

The scaled connectivity of different PGFC groupings between caries and caries-free PSCNs was compared using the union of PGFCs in caries and caries-free HCPCs. Statistically significant differential connectivity was observed when grouping PGFCs by taxonomic level (N=7 PGFCs enriched in PSCN_{Caries} and N=11 PGFCs enriched in PSCN_{Caries-free}) and none when grouped by functional level (FIG. 12). Although, the connectivity of high-level metabolic functional profiles is similar for both PSCNs, the taxa responsible for these driving functions are unique to the phenotype. The taxa with enriched connectivity in PSCN_{Caries} were Kingella_B oralis trailed by Granulicatella adiacens, Haemophilus_D parainfluenzae, Capnocytophaga leadhetteri, and Streptococcus oralis. The taxa with greatest enriched connectivity in PSCN_{Caries-free} were Streptococcus sanguinis, Abiotrophia sp001815873, an unclassified Neisseria, and Cardiobacterium hominis. Although unclassified Neisseria and Abiotrophia sp001815873 are enriched in PSCN_{Caries-free}, they are not present in the caries-free communities (FIG. 11E) because they were not in the caries-free HCPCs. This discrepancy in membership suggests that connectivities of these taxa, though enriched, were masked by other high connectivity taxa in PSCN_{Caries-free}.

Differential co-expression networks suggests community scale metabolic restructuring through Cardiobacterium hominis. Differential co-expression networks (DCN) reveal changes in connectivity between a reference (caries free) and treatment (caries) network. As ensemble PSCNs are the building blocks of DCNs, the DCNs provide the same benefits with respect to outlier resistance. Previous approaches have used DCNs but did not use compositionally-aware association metrics or ensemble networks (21, 35). While differential abundance/expression analyses can be useful in identifying feature enrichment (e.g., OTU, MAG, ORF, gene, etc.), each method has their own caveats in assumptions about the data distributions (well characterized in (36) with the establishment of reference frames) and provide no information regarding differences in pairwise interactions; an essential perspective when studying diseases resulting from dysbiosis. Using the PSCN_{Caries-free} as a reference network and PSCN_{Caries} as the treatment network, a DCN using the 875 PGFCs was constructed from the community detection analysis for seamless cross-referencing between PSCNs and the DCN. In the DCN, differential connectivity (denoted as Δk˜) is positive and negative when a connectivity is enriched in the caries and caries-free microbiomes, respectively. Unsupervised clustering of the DCN revealed 6 clusters, numbered 0-5 (FIG. 13A-C), of which there were 3 high connectivity DCN clusters (HCDC), each being diagnostic of phenotype; HCDC-6A.4 had enriched connectivity in the caries microbiome while HCDCs-6A.2 and 5 had enriched connectivity in the caries-free microbiome. For the only HCDC with connectivity enriched in the caries microbiome (HCDC-6A.4), the differential connectivity was primarily from Capnocytophaga sputigena, Kingella B oralis, Vellonella parvula A, Streptococcus sanguinis, Streptococcus oralis, and unclassified Streptococcus (BC82, BC101, BC125) (FIG. 16A) via carbohydrate and cofactor/vitamin metabolism (FIG. 16B).

HCDC-6A.4 included 43% of all taxa within the DCN. HCDCs with enriched connectivity in caries-free microbiome contained a broader range of microbes. However, most of these taxa were in HCDC-6A.2 with more than 77% of the taxa in the DCN which was not the case for HCDC-6A.5 with 40% of the taxa. In HCDC-6A.2, most of the differential connectivity was attributable to Streptococcus sanguinis, Abiotrophia sp001815873, an unclassified Neisseria (BC6), Rothia dentocariosa, and several Fusobacteriota via carbohydrate metabolism, ATP synthesis, carbon fixation, and cofactor/vitamin biosynthesis (FIG. 16B). While HCDC-6A.2 is heterogenous in terms of taxa membership, HCDC-6A.5 is fairly homogenous with most of the connectivity from Cardiobacterium hominis via carbohydrate metabolism. Veillonella parvula_A and Fusobacterium polymorphum are the only taxa that had membership in all HCDCs. As Veillonella parvula_A had high differential connectivity in both caries and caries-free phenotypes through different metabolic pathways, this finding may provide insight into dysbiosis. Several other taxa such as Haemophilus_D parainfluenzae, Fusobacterium polymorphum, Firmicutes, Capnocytophaga sputigena, and Rothia dentocariosa had membership in the HCDC-6A.4 and with at least one HCDC with negative differential connectivity (FIG. 16A).

Comparing set membership between HCDCs revealed key metabolic differences between microbiomes. HCDC-6A.4 exclusively had 14 KEGG modules with the most notable including pentose phosphate pathway, phosphate acetyltransferase-acetate kinase, beta-Lactam resistance, several cofactor/vitamin pathways. HCDC-6A.2 had 19 KEGG modules not in HCDC-6A.4 which include many carbohydrate metabolic pathways, crassulacean acid metabolism, reductive pentose phosphate cycle, formaldehyde assimilation, dissimilatory nitrate reduction, and biotin biosynthesis. HCDC-6A.5 only had 4 exclusive KEGG modules including citrate cycle, fumarate reductase, and Raetz pathway with citrate cycle and fumarate reductase metabolism from Cardiobacteriun hominis.

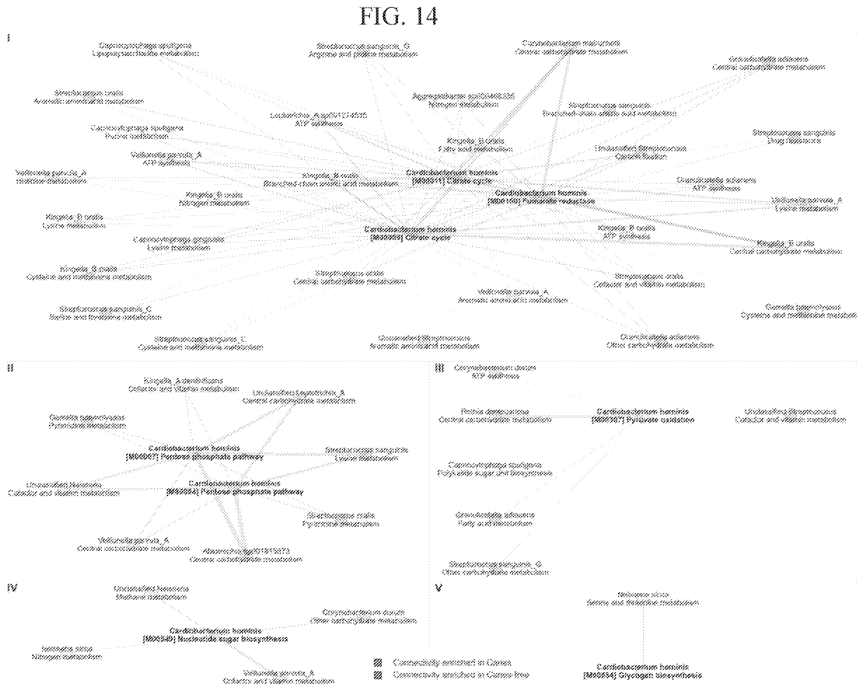

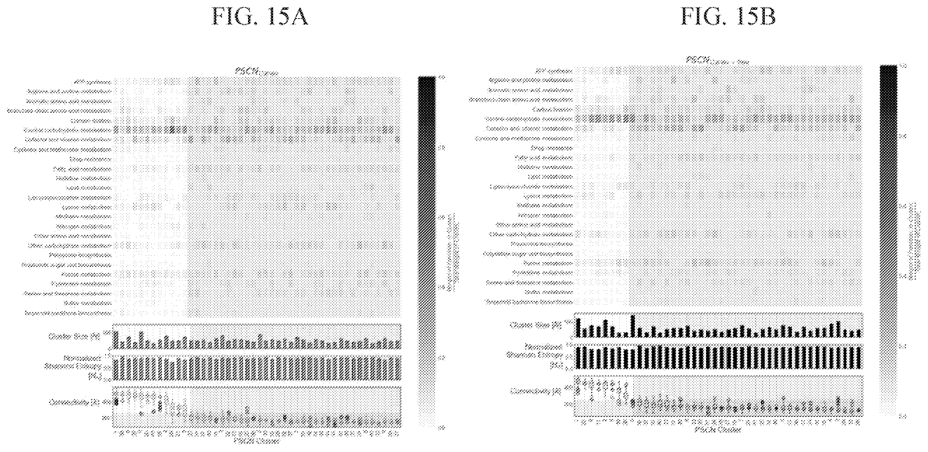

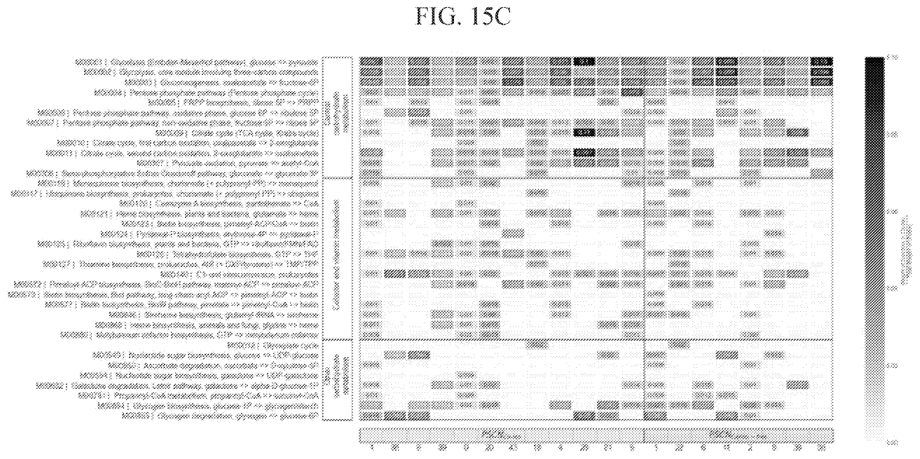

Hive plots are a network visualization framework that allows grouping nodes with respect to predefined axes. In this case, grouping PGFCs by taxa or KEGG categories for higher-level nodes and HCDCs for axes. The hive structure visualizes both intra- and inter-cluster differential connectivity clearly revealing hub nodes connecting clusters (FIGS. 13B-C). In the context of this DCN, Cardiobacterium hominis was a link between the highest differential connectivity HCDCs for caries (HCDC-6A.4) and caries-free (HCDC.6A.2) microbiomes even though each cluster's intra-cluster connectivity is sign specific. HCDC-6A.4 had very low connectivity to HCDC-6A.2 but both have high connectivity to HCDC-6A.5 primarily via Cardiobacterium hominis. However, positive differential connectivity from HCDC-6A.5 to HCDC-6A.4 was mainly from Cardiobacterium hominis carbohydrate metabolism and ATP synthesis to Kingella_B oralis, Capnocytophaga sputigena, Granulicatella adiacens, Corynebacterium matruchotii, and Veillonella parvula_A. In the connection between Cardiobacterium hominis and HCDC-6A.2, many more taxa was observed, also at greater differential connectivity magnitude, primarily through Streptococcus sanguinis, Abiotrophia sp001815873, and an unclassified Neisseria (BC6) with a long tail of taxa with negative differential connectivity. In this latter case, the highly negative differential connectivity from Cardiobacterium hominis to HCDC-6A.2 is spread out across many metabolic pathways and is not disproportionally weighted at carbohydrate and ATP synthesis suggesting Cardiobacterium hominis may have a holistic relationship in a caries-free microbiomes while also playing a potentially non-beneficial role in caries microbiomes. Accordingly, Cardiobacterium hominis carbohydrate metabolism and ATP synthesis modules were expanded out in a separate DCN (FIG. 14, Table 5).

TABLE 5
Carbohydrate metabolism and ATP synthesis nodes in DCN Leiden Communities. Category refers
to KEGG Level 3 metabolic category while description referrers to KEGG module description.
Community refers to Leiden communities for DCN. Abbreviations herein: ATP—ATP synthesis,
CCM—central carbohydrate metabolism, OCM—other carbohydrate mechanism.

| PGFCs | Species | Category | Description | HCDC | Community |
|---|---|---|---|---|---|
| BC123\|M00150 | Cardiobacterium | ATP | Fumarate reductase, prokaryotes | 5 | I |
| | hominis | | | | |
| BC46\|M00157 | Granulicatella | ATP | F-type ATPase, prokaryotes and | 4 | I |
| | adiacens | | chloroplasts | | |
| BC60\|M00144 | Kingella_{—}B oralis | ATP | NADH: quinone oxidoreductase, | 4 | I |
| | | | prokaryotes | | |
| BC21\|M00159 | Leptotrichia_{—}A | ATP | V-type ATPase, prokaryotes | 2 | I |
| | sp001274535 | | | | |
| BC1\|M00153 | Veillonella | ATP | Cytochrome bd ubiquinol oxidase | 2 | I |
| | parvula A | | | | |
| BC123\|M00009 | Cardiobacterium | CCM | Citrate cycle (TCA cycle, Krebs | 5 | I |
| | hominis | | cycle) | | |
| BC123\|M00011 | Cardiobacterium | CCM | Citrate cycle, second carbon | 5 | I |
| | hominis | | oxidation, 2-oxoglutarate => | | |
| | | | oxaloacetate | | |
| BC0b\|M00003 | Corynebacterium | CCM | Gluconeogenesis, oxaloacetate | 4 | I |
| | matruchotii | | fructose-6P | | |
| BC0b\|M00001 | Corynebacterium | CCM | Glycolysis (Embden-Meyerhof | 4 | I |
| | matruchotii | | pathway), glucose => pyruvate | | |
| BC0b\|M00002 | Corynebacterium | CCM | Glycolysis, core module involving | 4 | I |
| | matruchotii | | three-carbon compounds | | |
| BC46\|M00007 | Granulicatella | CCM | Pentose phosphate pathway, non- | 4 | I |
| | adiacens | | oxidative phase, fructose 6P => | | |
| | | | ribose 5P | | |
| BC60\|M00011 | Kingella B oralis | CCM | Citrate cycle, second carbon | 4 | I |
| | | | oxidation, 2-oxoglutarate | | |
| | | | oxaloacetate | | |
| BC60\|M00003 | Kingella_{—}B oralis | CCM | Gluconeogenesis, oxaloacetate | 4 | I |
| | | | fructose-6P | | |
| BC60\|M00001 | Kingella B oralis | CCM | Glycolysis (Embden-Meyerhof | 4 | I |
| | | | pathway), glucose => pyruvate | | |
| BC60\|M00002 | Kingella B oralis | CCM | Glycolysis, core module involving | 4 | I |
| | | | three-carbon compounds | | |
| BC60\|M00307 | Kingella B oralis | CCM | Pyruvate oxidation, pyruvate => | 4 | I |
| | | | acetyl-CoA | | |
| BC60\|M00308 | Kingella B oralis | CCM | Semi-phosphory lative Entner- | 4 | I |
| | | | Doudoroff pathway, gluconate => | | |
| | | | glycerate-3P | | |
| BC36\|M00005 | Streptococcus oralis | CCM | PRPP biosynthesis, ribose 5P => | 4 | I |
| | | | PRPP | | |
| BC46\|M00854 | Granulicatella | OCM | Glycogen biosynthesis, glucose- | 4 | I |
| | adiacens | | 1P => glycogen/starch | | |
| BC18\|M00157 | Abiotrophia | ATP | F-type ATPase, prokaryotes and | 2 | II |
| | sp001815873 | | chloroplasts | | |
| BC18\|M00159 | Abiotrophia | ATP | V-type ATPase, prokaryotes | 2 | II |
| | sp001815873 | | | | |
| BC123\|M00004 | Cardiobacterium | CCM | Pentose phosphate pathway | 5 | II |
| | hominis | | (Pentose phosphate cycle) | | |
| BC123\|M00007 | Cardiobacterium | CCM | Pentose phosphate pathway, non- | 5 | II |
| | hominis | | oxidative phase, fructose 6P => | | |
| | | | nbose 5P | | |
| BC1\|M00003 | Veillonella | CCM | Gluconeogenesis, oxaloacetate => | 5 | II |
| | parvula A | | fructose-6P | | |
| BC1\|M00001 | Veillonella | CCM | Glycolysis (Embden-Meyerhof | 5 | II |
| | parvula A | | pathway), glucose => pyruvate | | |
| BC1\|M00002 | Veillonella | CCM | Glycolysis, core module involving | 5 | II |
| | parvula A | | three-carbon compounds | | |
| BC123\|M00854 | Cardiobacterium | OCM | Glycogen biosynthesis, glucose- | 5 | III |
| | hominis | | IP => glycogen/starch | | |
| BC123\|M00307 | Cardiobacterium | CCM | Pyruvate oxidation, pyruvate => | 5 | IV |
| | hominis | | acetyl-CoA | | |
| BC123\|M00549 | Cardiobacterium | OCM | Nucleotide sugar biosynthesis, | 5 | V |
| | hominis | | glucose => UDP-glucose | | |

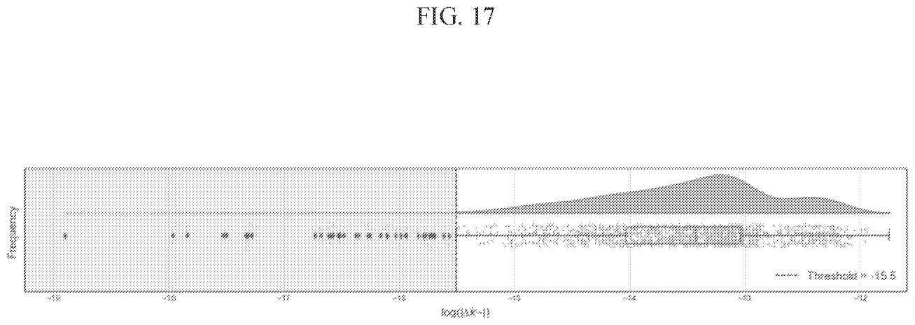

After removing low connectivity edges (FIG. 17), this DCN revealed 5 Leiden communities, denoted as Communities-7.I-V, with the 2 largest communities being Community-7.I and Community-7.HI. Consistent with previous hive networks, a community with connectivity primarily enriched in the caries microbiome (Community-7.I) and several communities with connectivity almost exclusively enriched in caries-free microbiome (Communities-7.II-V) was observed. The exception to the latter is Cardiobacterium hominis pyruvate oxidation and Capnocytophaga sputigena polypeptide sugar unit biosynthesis connectivity enriched in caries microbiome in Community-7.III. Community-7.II, the largest of the negative differential connectivity communities, was far less complex than Community-7.I and has Cardiobacterium hominis pentose phosphate as highly central nodes. There were 3 other small communities with negative differential connectivity and the most interesting of these is Community-7.IV as Cardiobacterium hominis glucose to UDP-glucose conversion is connected to mostly to Veillonella parvula_A cofactor/vitamin metabolism but also Neisseria nitrogen metabolism/methane metabolism and Corynebacterium durum carbohydrate metabolism.

The most complex and informative community is Community-7.I, which is primarily composed of positive differential connectivity edges, those enriched in the caries microbiome. The negative differential connectivity edges are primarily from ATP synthesis of Veillonella parvula_A and Leptotrichia_A sp001274535. Said nodes only have negative differential connectivity edges which suggest they are influential to the rest of the community in a caries-free microbiome and this may provide insight into community-scale restructuring in the caries microbiome. Without being bound by theory, the only nodes with both positive and negative differential connectivity edges are from Cardiobacterium hominis supporting the hypothesis that Cardiobacterium hominis is an essential player in the transition from caries-free to caries phenotypes and vice versa. However, the most striking feature of this community is that Cardiobacterium hominis citrate cycle and fumarate reductase are highly centralized suggesting a shift in carbohydrate metabolism from pentose phosphate cycle to citrate acid cycle in the caries microbiome. Various types of carbohydrate metabolism in Community-7.1 with positive differential connectivity from several other organisms were observed (Table 5).

Predictive models applied to caries diagnosis Feature selection and predictive modeling was implemented to further evaluate PGFC features that were indicative of caries diagnosis. In particular, the Clairvoyance feature selection algorithm (37) that has been previously evaluated on identifying diagnostic genes related to antibiotic resistance (37) and multimodal associations related to childhood undernutrition (38) was used to identify PGFCs that were able to accurately discriminate caries individuals from caries-free individuals. To allow for seamless interpretation with the network analysis, the set of 212 PGFCs from the DCN were used as input into the feature selection algorithm and this was implemented for PGFCs represented as MCR and as CLR transformed abundances to yield two separate feature sets. This mixed feature architecture allowed for a novel type stacking ensemble where each base classifier uses a specific feature set and feature representation (e.g., MCR and CLR values simultaneously) leveraging the strength of each measurement in the ability to predict caries phenotype. The MCR feature set included 36 PGFCs, the CLR feature set included 27 PGFCs, and 11 PGFCs were shared between both base models (FIG. 18B). The PGFCs selected via feature selection included Cardiobacterium hominis pentose phosphate and TCA cycle as some of the highest weighted features that were able to discriminate caries phenotypes. The stacking ensemble classification model was able to predict unobserved twin groupings with an accuracy of >99% (see Methods for cross-validation). In this context, accuracy can be interpreted as the reliability of a feature set to be sufficient in diagnosing caries.

### Example 2

Cluster genomes into SLCs based on sequence similarity. For each SLC, utilize protein clustering to generate SLC-specific orthogroups. Determine KEGG orthology for each of the SLC-specific orthogroups, and map metatranscriptomic reads to the orthogroups. The relative abundance of taxon specific orthogroups will serve as the primary dataset.

Group the gene expression counts by the SLC-specific KEGG modules into PGFCs. At this point, a table that is organized as n samples by m PGFCs. PGFCs with low MCR's (e.g., MCR<50%) are filtered out. The MCR indicates what % of KEGG modules are detected for the KEGG module to be executed.

A differential co-expression analysis of PGFCs is conducted by taking PSCNs and then subtracting them from each other to build a DCN. Hierarchical clustering and plotting the differential connectivity is employed to determine which clusters of PGFCs have the highest differential connectivity. Those with the highest differential connectivity are most predictive of the caries phenotype. Based on this particular feature, clusters that are highly differentially connected based on empirical (data-derived) metrics are used. This subset of PGFCs expression matrix is used an input into the Clairvoyance feature selection process. The Clairvoyance feature selection proceed feeds into a predictive model and cross validation exercises.

### Methods

Study design and patient cohort overview, used for metatranscriptomics. The primary objective of this study was to characterize the supragingival dental plaque of children with and without caries. A secondary objective assess how host genetics affects the microbiome in relation to caries and was not prioritized in this study due to the environmental effects on transcription. This study has been analyzed using 16S rRNA amplicon (13, 50) and shotgun metagenomics (27, 28) datasets. The metatranscriptomics dataset herein has not been analyzed prior to this study. Our metagenomics dataset contains 87 subjects and Our metatranscriptomics dataset contains 91 subjects with an overlap of 26 subjects. For each subject, there is a single sample per omics technology.

The study design has been described previously for this BioProject (www.ncbi.nlm.nih.gov/bioproject/?term=PRJNA383868) in sister studies (13, 27, 50). In particular for the metatranscriptomics cohort, dental plaque samples were collected from participants of the University of Adelaide Craniofacial Biology Research Group Tooth Emergence and Oral Health Study (CBRG) (n=52), and the Murdoch Children's Research Institute (MCRI) Peri/Postnatal Epigenetic Twins Study (PETS) (n=39). Human research with PETS subjects was approved by the Royal Children's Hospital Human Research Ethics Committee (#3174), and the CBRG cohort was approved by The University of Adelaide Human Research Ethics Committee (#H-2013-097). Research at the J. Craig Venter Institute was approved by the JCVI Institutional Review Board (#2013-182). All research was performed according to the listed institutions guidelines and informed consent was obtained from all participants' parent and/or legal guardians. Inclusion criteria included 5-11-year-old twins whose parents consented to this portion of the study.

Supragingival plaque samples were obtained at the commencement of a dental examination. Prior to sample collection, participants were guided not to brush their teeth the night preceding the sample collection nor on the day of sample collection. Metadata were collected from three separate questionnaires completed by the parents during the period from consent to prior to the dental examination being undertaken. The clinical questionnaires consisted of a total of 132 questions to survey oral and medical health, dietary patterns, and development patterns, and dental hygiene. Visual inspection of the oral cavity followed International Caries Detection and Assessment System (ICDAS II). The ICDAS II was used to assess and define dental caries at the initial and early enamel lesion stages through to dentin and more advanced stages of the disease. Examiners were experienced clinicians who had undergone rigorous calibration and were routinely recalibrated across measurement sites to minimize error. Caries history in each participant was initially reduced to a whole-mouth score and three classifications were utilized. no evidence of current or previous caries experience; evidence of current caries affecting the enamel layer only on one or more tooth surfaces; evidence of previous or current caries experience that has progressed through the enamel layer to involve the dentin on one or more tooth surfaces (including restorations or tooth extractions due to caries). For the purpose of phenotypic analysis, disease states from twins were classified as evidence of caries in enamel or dentin.

Assembly, classification, dereplication for metagenome assembled genomes (bacteria and viruses). A metapantranscriptomics approach was pursued by assembling and binning each metagenome separately, performing species-level clustering, and a within SLC orthology analysis (FIG. 7A). The primary aim of this research is to investigate inferred interactions within the supragingival plaque microbiome and how these interactions are associated with either caries or caries-free phenotypes. With this scope in mind, only the conserved regions of each SLC were analyzed and, when mapping reads, produced a denser matrix that is better suited for network analysis in identifying core patterns.

The metagenomics dataset contains 87 subjects and were characterized previously using a co-assembly approach (27). Using updated state-of-the-art consensus methodologies, this dataset was revisited to provide higher quality MAGs and annotations resulting in more accurate biological interpretations. Each of these 87 processed metagenomic samples were assembled on a per sample basis using SPAdes v3.15.2(metaSPAdes mode, Nurk et al., 2017) and binned using several binning algorithms including: 1) MaxBin2 v2.2.7 (52), 2) MetaBAT2 v2.15 (53), 3) VAMB v3.0.2 (54), and 4) VAMB using “multi-split binning” which uses all samples together. The binning assignments were refined using DAS Tool v1.1.2 (55) yielding 833 MAGs that were classified using GTDB-Tk v1.5.0 (reference database: r202; Chaumeil et al., 2020) indicating all MAGs were bacteria and none were archaea. The 883 bacteria MAGs were assessed using the lineage_wf pipeline of CheckM v1.1.3 (57) and 658 of which were of at least medium quality according to the guidelines established by the Genomic Standard Consortium (>50% completeness and <10% contamination; Bowers et al., 2017) which are referred to here after as Assembly_{Bacteria}. For the Patescibacteria CPR, the 43 marker genes (cpr_43_markers.hmm) were used as proposed by Brown et al. 2015 (59). These 124 of the 658 MAGs had no confident species-level classification from GTDB-Tk and indicate novel species from known genera.

Contigs, both binned and unbinned, that were not included in Assembly_{Bacteria} were input into VirSorter2 v2.2.2 (-include-groups dsDNAphage, NCLDV, ssDNA, lavidaviridae) (60) and quality assessed using CheckV v0.8.1 (reference database: v1.0) (61) yielding 179 DNA viruses of at least medium quality; referred to as Assembly_{VirusDNA}. The recommended cutoffs in CheckV for considering viral contigs are the following; 1) number of viral genes >0; 2) the number of viral genes≥5 times the number of host genes; 3) completeness≥50%; 4) checkv_quality and miuvig_quality are above medium quality (62). Reads from the 91 metatranscriptomic samples that did not map to any of the metagenomic contigs (both binned and unbinned) were assembled using SPAdes (rnaSPAdes mode) on a per sample basis. These transcript assemblies were input into VirSorter2 (-include-groups RNA) and assessed for quality using CheckV yielding 10 RNA viruses; referred to as Assembly_{VirusRNA}. Classifications for viruses were assigned using the aai_top_hit field from completeness.tsv where RefSeq identifiers were propagated and DTR_[XXXXXX] identifiers were extrapolated from the genome_db/checkv_circular.tsv file of the CheckV database. Only non-proviruses were used in downstream analysis to prevent host bias.

Dereplication of redundant species were clustered using FastANI v1.32(63) with a cutoff of ≥95% ANI and connected components were determined using NetworkX v2.5 (64). FastANI clustering was performed separately on the following: 1) Assembly_{Bacteria} resulting in 135 unique clusters, prefixed by BC, with 64 of the clusters representing singleton clusters of size 1; and 2) Assembly_{VirusDNA}+Assembly_{VirusRNA} resulting in 142 unique clusters, prefixed by VC, with 113 singleton clusters. Manual curation of FastANI defined clusters was performed when the GTDB-Tk classifications were clearly split (i.e. FastANI clusters: BC0, BC11, and BC25). These FastANI clusters are referred to in this example as species-level clusters (SLC).

The 289,342 contigs from Assembly_{Bacteria} (658 bacterial MAGs), Assembly_{VirusDNA} (179 DNA viruses), and Assembly_{VirusRNA} v4 (10 RNA viruses) were combined into the Assembly_{Catalogue} that is used for all read mapping, gene modeling, and statistical analysis. A flowchart for bacterial, viral, and orthogroup pipelines are available in FIGS. 6A-C.

Gene models, functional annotation, and orthology analysis. Gene models were created for Assembly_{Catalogue} using Prodigal v2.6.3 (-meta mode; Hyatt et al., 2010) generating putative proteins and GFF files used for annotations and read mapping. The putative proteins were annotated using both Diamond v2.0.8 (66) against RefSeq's non-redundant database (accessed 2020.04.02) KOFAMSCAN v1.3.0 (reference database accessed 2020.05.28; Aramaki et al., 2020).

Orthology analysis was performed for each putative proteins in each FastANI cluster using OrthoFinder v2.5.2 (68). For singleton FastANI clusters, dummy genomes were provided to yield a paralog-style analysis to stay consistent with the orthogroup analysis where intra-genome orthogroups are permitted. Consensus annotations for were determined for each orthogroup using the annotations from Diamond and KOFAMSCAN.

Read preprocessing and mapping to genome catalogue. HISAT2 v2.2.1 (69) was used for all read mapping to Assembly_{Catalogue} for both metagenomics and metatranscriptomics datasets. Read counts tables were constructed with feature Counts v2.0.1 (70) using the bam files generated from HISAT2, the Assembly_{Catalogue} fasta, and the gene models defined using Prodigal. Counts for FastANI clusters or MAGs were computed by summing contig-level read counts using a SAF file generated from Assembly_{Catalogue}.

Reads were preprocessed using KneadData v0.6.1 by quality trimming using Trimmomatic v0.39 (71), aligning reads to the human genome (GRCh37.p4) via Bowtie2 v2.4.3 (72) bow, and removing aligned reads for assembly and mapping. The metagenomics and metatranscriptomics datasets contained reads with a post-processed sequencing depth of 2,946,448±1,432,642 reads and 5,963,060±1,726,024 reads, respectively. The alignment rate to Assembly_{Catalogue} for metagenomics was 73.0±7% and 69.7±5.9% for metagenomics and metatranscriptomics datasets, respectively. The missing alignments are due to the fact that the reads were mapped to the highest quality bacterial MAGs and viral contigs.

Phylogenomic functional categories. Within the dataset, there are 255,737 SLC-specific orthogroups which would result in ˜32.7 billion non-redundant connections in a fully-connected coexpression network; an insurmountable dataset for exploratory analysis on most modern machines. Instead of using draconian filtering thresholds of orthogroups, a feature engineering technique was devised that would allow seamless transitions from read↔ORF/orthogroup↔contig/MAG/SLC↔engineered feature using custom taxonomy fields and functional assignments (e.g. KEGG, MetaCyc, PFAM).

With this in mind, the PhyloGenomic Functional Category (PGFC) is presented as a supervised microbiome feature engineering method that can be used for high-level statistical analysis and unpacked back into individual orthogroups (or ORFs) unlike dimensionality reduction methods such as PC[o]A, t-SNE, or UMAP. PGFCs essentially group low-level features, orthogroups in this context, by a taxonomic unit (SLC) and a functional unit (KEGG module) (FIG. 7B), distinguishable to HUMAnN (73, 74) which does not allow the flexibility for custom low-level features from de-novo meta-omics. Another similar approach is the amalgam where compositions can either have exclusive or non-exclusive mappings between the original feature and engineered feature (75). However, these engineered features cannot be collapsed and expanded with respect to predefined categories such as taxonomy and metabolism so they were not explored.

PGFCs are composite features that group metabolic functional information with genome-resolved taxonomy assignments and were created by grouping all of the orthogroups that had KEGG orthology, defined via KOFAMSCAN, and extending the grouping up the hierarchy to modules with respect to taxonomy. Taxonomy for PGFCs were assigned to the FastANI cluster of origin. PGFCs were implemented using our EnsembleNetworkX v2021.06.24 Python package (38, 76) via the CategoricalEngineeredFeature class.

A strict filtering scheme was implemented to identify only the most robust patterns. MCRs were calculated from the KEGG orthologs defined by KOFAMSCAN using MicrobeAnnotater v2.0.5 (77). PGFCs were removed if they did not have MCR >50% in at least 20 samples resulting in 2,478 PGFCS representing 89 bacterial taxonomic units and 113 functional units from 8554 orthogroups. The rationale behind using such a stringent threshold is that many downstream associations may be misleading if only a small number of enzymes are represented in the module.

The unfiltered PGFC dataset contains 15,125 PGFCs incorporating 171 SLCs and 267 KEGG modules. Although this dataset was relatively dense, many of the KEGG modules were incomplete with respect to a SLC in a sample. For increased biological interpretability, PGFCs with KEGG modules that were largely incomplete, MCR<0.5, were filtered out to identify patterns with higher confidence amongst these high-level features; a feature, to our knowledge, is not implemented in similar methodologies. The MCR-filtered PGFC dataset contained 2,478 PGFCs representing 89 taxonomic units and 113 functional units from 8,554 orthogroups; all of which are from bacterial SLCs. In orthogroup-space, these features would amount to ˜37 million non-redundant connections but ˜3 million % in PGFC-space effectively compressing the information content by ˜92%; making prototyping and data exploration tractable on modern compute machines.

The PGFC counts were normalized via prevalence by scaling the counts by the total number of ORFs in each sample and in each PGFC. Our dataset prior to preprocessing produced 15,125 PGFCs representing 171 bacterial and viral taxonomic units and 267 functional units from 24,640 orthogroups.

Differential expression analysis. Several differential abundance packages were tested including, but not limited to, ALDEx2 v1.12.0 (78, 79) and ANCOM v0.5.6 (80, 81). These analyses were performed at 2 levels: 1) summing contig counts per SLC as a measure of total taxonomic expression in a sample; and 2) the preprocessed PGFC feature table used as input in network analysis. For ALDEx2, Welch's t-test, Wilcoxon test, Kruskal-Wallace, and GLM tests with “iqlr”, “zero”, and “all” denominators were tried. For ANCOM, alpha 0.5, tau 0.02, and theta 0.1 parameters were tried.

Typically, expression:abundance ratios are calculated using log fold-change of relative abundances. For consistency with our compositional approach, differential RNA and DNA abundance were calculated by taking the difference in CLR between biosamples with statistical significance calculated via Mann-Whitney U-test and Benjamini/Hochberg adjusted p-values. As the CLR transform is in log-space, the difference between RNA and DNA values are akin to log fold-change.

Ensemble networks and connectivity measurements. Ensemble co-expression networks were designed to robustly model associations within a system and differences in associations between systems such as phenotypes. In co-expression networks, it is assumed that weighted associations between features estimate biologically meaningful interactions within a system. Instead of creating a single cross-sectional network using all of the samples at once, ensemble networks were implemented by bootstrapping samples, with or without repetition, and calculating a distribution of weighted associations as the basis for each edge in the networks. Ensemble machine-learning approaches have higher performance than singular methods (82) and boosting has been shown to resistant to outliers (83) by minimizing the influence of individual samples and, in the context of networks, performed by representing edges as a distribution rather than a singular value.

For pairwise connectivity measurements, rho proportionality (14, 16, 17, 84) was implemented, which is a compositionally-aware association matrix akin to correlation. Rho proportionality is in the range [−1,1] where −1 means inversely proportional and 1 is perfectly proportional. Edge connectivity refers to the median rho proportionality associations from our ensemble networks and serve as the foundation for all higher-level connectivity measurements such as node connectivity, intra/inter-community, etc. For interpretability, only the positive associations were analyzed as these have the most direct biological meaning.

Node connectivity is implemented as weighted-degree and is computed by summing all weighted edges connected to a particular node. Similar to the node connectivity calculations, connectivity with respect to other higher-level groupings such as taxonomy, cluster, or metabolic function was investigated by summing all of the non-self connectivities within that grouping. Differential connectivity is in reference to the caries-free cohort so that a positive DCN connectivity shows an enrichment in connectivity in the caries cohort while a negative connectivity shows a depletion in connectivity within the caries cohort.

Connectivity in this study is represented as either unscaled connectivity [k] or scaled connectivity [k˜] which is computed by scaling to total network connectivity in that the sum of scaled connectivities equals 1 in a network. Scaled connectivity is performed at the level of edges, that is all summed edge connections equal 1, and are useful when comparing networks with different total connectivity.

Phenotype-specific co-expression networks (PSCNs). Differences in connectivity between caries and caries-free PSCNs were investigated using PGFCs as nodes and rho proportionality as the metric for associations (N_{nodes}=2,478; N_{edges}=3,069,003). These associations are interpreted as inferred interactions with insight into potential phenotype-specific metabolism coupled to taxonomy. Our ensemble co-expression network analysis is derived from the following network states: (1) caries cohort co-expression; and (2) caries-free cohort co-expression. After preprocessing as described previously, PSCNs were constructed using the following operations: (1) normalize the PGFC counts by dividing the summed counts by number of detected orthogroups in each sample relative to the PGFC grouping; (2) subset the data by phenotype; (3a) pseudo-random selection of 90% of the samples, (3b) pairwise rho proportionality for compositionally-aware associations among PGFCs within subject subset; (3c) stack non-redundant edge weights in array; (3d) repeat steps 3a-3c for 500 iterations with consecutive random seeds for reproducibility; (4) compute the median and median absolute deviation for each edge distribution as a representative edge weight; (5) split the positive associations (A+) and negative associations (A−) separately where A represents the association matrix; and (6) use the adjacency matrices for weighted edges to build fully-connected networks via NetworkX v2.4. For interpretability, only the positive associations were used in downstream network analysis. Normalized entropy for measuring network cluster homogeneity was computed using the following equation: Hₙ(p)=−Σᵢ pᵢ log₂ pᵢ/log₂ n, where n is the number genera in the cluster and pᵢ is the weighted proportion of genus i in the cluster.

Differential Network Analysis. Our approach for quantifying the differences between network states is implemented using the following network types: (1) phenotype-specific coexpression networks (PSCN); and (2) differential co-expression networks (DCN). For PSCNs, there are networks that share the same nodes but differ in their edge weights. For instance, PGFC-level PSCNs have 2,478 PGFC nodes for both caries and caries-free systems. This property allows us to easily create DCNs from PSCNs by taking the difference between adjacency networks as follows: PSCN_{Caries}−PSCN_{Caries-free}=DCN.

To determine whether groupings such as taxonomy, metabolic function, and cluster have statistically different connectivities between caries and caries-free PSCNs, Wilcoxon signed rank-tests were used (SciPy v1.4.1) (85). For Wilcoxon signed-rank tests, only groups that had n>20 PGFCs were tested as recommended by the SciPy documentation. Benjamini/Hochberg multiple test correction was used to compute FDR values from Wilcoxon signed-rank test p-values.

Unsupervised network clustering and community detection. Many unsupervised methods can use a precomputed distance matrix as input. To seamlessly feed these networks into these unsupervised methods, network adjacency matrices were converted to dissimilarity networks and used these as the precomputed distance matrix. As mentioned, these networks were split into positive and negative networks for downstream analysis. However, signed associations for clustering were used to maximize the information content used for the most informative clustering. To transform this signed association into a dissimilarity matrix that preserves all information content in the original association matrix, the formula 1-rho+ was used, which is an adaptation of correlation distance but applied to rho proportionality.

For unsupervised clustering, the following methods were used: (1) agglomerative hierarchical clustering of the dissimilarity network with ward linkage (SciPy v1.4.1) (85) with hybrid methods for cutting dendrograms (dynamicTreeCut v1.63.1); and (2) ensemble Leiden community detection of networks (30). Community detection does not necessarily reference bona fide microbial communities but more specifically communities of nodes within a network. Ensemble Leiden community detection was performed by running the Leiden algorithm (leidenalg v0.8.3) using 1000 different random seeds and grouping nodes that were in the same community for all iterations.

Agglomerative hierarchical clustering-based clusters are named by their respective figure and integer label. For example, Cluster-2.5 refers to cluster 5 of FIG. 9, HCPC-4A.1 refers to high connectivity PSCN cluster 1 of FIG. 11A, and HCDC-6A.2 refers to high connectivity differential connectivity cluster 2 of FIG. 13A. Leiden communities are named by their respective figure, panel, and roman numeral. For example, Community-4C.I is Leiden community I of FIG. 11C.

Feature selection and predictive modeling. The Clairvoyance feature selection algorithm (7) was used to identify features that could discriminate caries phenotypes. The feature set used as input into the feature selection algorithm was the 212 PGFCs used in the DCN analysis. Of this feature set, two separate feature representations were used: 1) MCR; and 2) CLR where MCR refers to module completion ratios of PGFCs and CLR refers to center log-ratio transformed abundances. This implementation of stacking uses custom feature sets for each base classifier whose prediction probabilities are modeled using a meta classifier (FIGS. 18A-B). These feature sets and their respective base classifiers were combined into a stacking classifier implemented in sklearn v0.22.2 composed of clf_{MCR} (LogisticRegression(C=0.474, penalty=L2) and clf_{CLR} (LogisticRegression(C=0.421, penalty=L2) where clf refers to a base estimator whose output probabilities are used as input into a meta-classifier (RidgeClassifier(alpha=0.8)), C, penalty, and alpha refer to algorithm hyperparameters. The evaluation strategy used was Leave Family Out Cross Validation (LFOCV) accuracy, where family refers to the unique family identifier that corresponds to each twin or triplet pair, simulating predictive performance on new subjects.

Visualization. Traditional network plots were implemented using a combination of NetworkX v2.4 for plotting with graphviz layout for graphical layout and Matplotlib as the backend. Additionally, the neato layout algorithm from graphviz was used, which creates virtual physical models and runs an iterative solver to find low energy configurations. Hive plots were implemented using the Hive class from HiveNetworkX v2021.05.18 (46, 86). Dendrograms were implemented using the Agglomerative class from soothsayer v2021.05.18 (37, 87). Heatmaps and clustermaps were implemented using seaborn v0.9. All other figures were generated with Matplotlib v3.2.3 in Python v3.8.2 unless specifically noted otherwise.

Furthermore, the methods provided a reproducible pipeline for clustering sample-specific genomes that integrates metagenomics and metatranscriptomics analysis regardless of biosample overlap. Methods related to species-level clustering were developed to characterize biologically accurate MAGs across non-overlapping samples. Further, novel feature engineering and network analysis techniques were developed to reveal the regime shifts between caries and caries-free microbiomes. These techniques revealed how regime shifts occur when certain taxa switch their interactions with other organisms and metabolic pathways. Such regime shifts may be utilized to serve as predictor of caries microbiomes.

### Additional Details

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums). Computer readable storage mediums may also be referred to herein as computer readable storage or computer readable storage devices.

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a service, module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, algorithms, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERENCES

1. S. L. James, et al., Global, regional, and national incidence, prevalence, and years lived with disability for 354 Diseases and Injuries for 195 countries and territories, 1990-2017: A systematic analysis for the Global Burden of Disease Study 2017. Lancet 392, 1789-1858 (2018).
2. B. A. Dye, X. Li, E. D. Beltran-Aguilar, Selected oral health indicators in the United States, 2005-2008. NCHS Data Brief, 1-8 (2012).
3. M. A. Peres, et al., Oral diseases: a global public health challenge. Lancet 394, 249-260 (2019).
4. OECD, “Health at a Glance 2017: OECD Indicators” (OECD, 2017) doi.org/10.1787/health_glance-2017-en (Jun. 14, 2021).
5. L. T. Humphrey, et al., Earliest evidence for caries and exploitation of starchy plant foods in Pleistocene hunter-gatherers from Morocco. Proc. Natl. Acad. Sci. U.S.A 111, 954-959 (2014).
6. P. D. Marsh, Microbial Ecology of Dental Plaque and its Significance in Health and Disease. Adv. Dent. Res. 8, 263-271 (1994).
7. N. Takahashi, B. Nyvad, The role of bacteria in the caries process: ecological perspectives. J. Dent. Res. 90, 294-303 (2011).
8. I. Kleinberg, A mixed-bacteria ecological approach to understanding the role of the oral bacteria in dental caries causation: an alternative to Streptococcus mutans and the specific-plaque hypothesis. Crit. Rev. Oral Biol. Med. 13, 108-25 (2002).
9. P. D. Marsh, D. J. Bradshaw, Physiological approaches to the control of oral biofilms. Adv. Dent. Res. 11, 176-185 (1997).
10. C. Folke, et al., Regime shifts, resilience, and biodiversity in ecosystem management. Annu. Rev. Ecol. Evol. Syst. 35, 557-581 (2004).
11. B. Nyvad, N. Takahashi, Integrated hypothesis of dental caries and periodontal diseases. J. Oral Microbiol. 12 (2020).
12. S. R. Smith, et al., Transcriptional Orchestration of the Global Cellular Response of a Model Pennate Diatom to Diel Light Cycling under Iron Limitation. PLOS Genet. 12, e1006490 (2016).
13. A. Gomez, et al., Host Genetic Control of the Oral Microbiome in Health and Disease. Cell Host Microbe 22, 269-278.e3 (2017).
14. D. Lovell, V. Pawlowsky-Glahn, J. J. Egozcue, S. Marguerat, J. Bahler, Proportionality: A Valid Alternative to Correlation for Relative Data. PLOS Comput. Biol. 11, e1004075 (2015).
15. P. Langfelder, S. Horvath, WGCNA: an R package for weighted correlation network analysis. BMC Bioinformatics 9, 559 (2008).
16. I. Erb, C. Notredame, How should we measure proportionality on relative gene expression data? Theory Biosci. 135, 21-36 (2016).
17. T. P. Quinn, M. F. Richardson, D. Lovell, T. M. Crowley, Propr: An R-package for Identifying Proportionally Abundant Features Using Compositional Data Analysis. Sci. Rep. 7, 1-9 (2017).
18. T. P. Quinn, I. Erb, M. F. Richardson, T. M. Crowley, Understanding sequencing data as compositions: an outlook and review. Bioinformatics 34, 2870-2878 (2018).
19. J. T. Morton, et al., Balance Trees Reveal Microbial Niche Differentiation. mSystens 2, e00162-16 (2017).
20. J. L. Espinoza, N. Shah, S. Singh, K. E. Nelson, C. L. Dupont, Applications of weighted association networks applied to compositional data in biology. Environ. Microbiol. 22, 3020-3038 (2020).
21. T. F. Fuller, et al., Weighted gene coexpression network analysis strategies applied to mouse weight. Mamm. Genome 18, 463-472 (2007).
22. N. Altman, M. Krzywinski, The curse(s) of dimensionality. Nat. Methods 15, 399-400 (2018).
23. L. Van Der Maaten, Accelerating t-SNE using Tree-Based Algorithms. J. Mach. Learn. Res. 15, 1-21 (2014).
24. L. McInnes, J. Healy, J. Melville, UMAP: Uniform Manifold Approximation and Projection for Dimension Reduction (2018) (Sep. 10, 2019).
25. J. N. Paulson, O. C. Stine, H. C. Bravo, M. Pop, Differential abundance analysis for microbial marker-gene surveys. Nat. Methods 10, 1200-1202 (2013).
26. J. D. Silverman, K. Roche, S. Mukherjee, L. A. David, Naught all zeros in sequence count data are the same. Comput. Struct. Biotechnol. J. 18, 2789-2798 (2020).
27. J. L. Espinoza, et al., WGCNA: an R package for weighted correlation network analysis. M Bio 9 (2018).
28. A. Shaiber, A. M. Eren, Composite metagenome-assembled genomes reduce the quality of public genome repositories. M Bio 10, e00725-19 (2019).
29. V. D. Blondel, J. L. Guillaume, R. Lambiotte, E. Lefebvre, Fast unfolding of communities in large networks. J. Stat. Mech. Theory Exp., P10008 (2008).
30. V. A. Traag, L. Waltman, N. J. van Eck, From Louvain to Leiden: guaranteeing well-1147 connected communities. Sci. Rep. 9, 1-12 (2019).
31. F. Zheng, et al., HiDeF: identifying persistent structures in multiscale 'omics data. Genome Biol. 22, 1-15 (2021).
32. S. J. Wilson, A. D. Wilkins, C. H. Lin, R. C. Lua, O. Lichtarge, Discovery of functional and disease pathways by community detection in protein-protein interaction networks. Pacific Symp. Biocomput. 22, 336-347 (2017).
33. Y. X H, R. M, Multi-label classification and label dependence in in silico toxicity prediction. Toxicol. In Vitro 74 (2021).
34. M. A. Jackson, et al., Detection of stable community structures within gut microbiota co-1156 occurrence networks from different human populations. Peer J6 (2018).
35. C. L. Hsu, H. F. Juan, H. C. Huang, Functional Analysis and Characterization of Differential Coexpression Networks. Sci. Rep. 5, 13295 (2015).
36. J. T. Morton, et al., Establishing microbial composition measurement standards with reference frames. Nat. Commun. 10, 1-11 (2019).
37. J. L. Espinoza, et al., Predicting antimicrobial mechanism-of-action from transcriptomes: A generalizable explainable artificial intelligence approach. PLOS Comput. Biol. 17, el008857 (2021).
38. H. M. Nabwera, et al., Interactions between fecal gut microbiome, enteric pathogens, and energy regulating hormones among acutely malnourished rural Gambian children. E Bio Medicine 73, 103644 (2021).
39. L. Chen, et al., Gut microbial co-abundance networks show specificity in inflammatory bowel disease and obesity. Nat. Commun. 11, 1-12 (2020).
40. X. Yang, L. He, S. Yan, X. Chen, G. Que, The impact of caries status on supragingival plaque and salivary microbiome in children with mixed dentition: a cross-sectional survey. BMC Oral Heal. 21, 1-13 (2021).
41. K. B J, et al., Pyrosequencing analysis of the oral microflora of healthy adults. J. Dent. Res. 87, 1016-1020 (2008).
42. E. Zaura, B. J. Keijser, S. M. Huse, W. Crielaard, Defining the healthy “core microbiome” of oral microbial communities. BMC Microbiol. 9, 259 (2009).
43. W. F. Liljemark, et al., Distribution of oral Haemophilus species in dental plaque from a large adult population. Infect. Immun. 46, 778 (1984).
44. S. Amat, H. Lantz, P. M. Munyaka, B. P. Willing, Prevotella in Pigs: The Positive and Negative Associations with Production and Health. Microorganisms 8, 1-27 (2020).
45. U. T. N, et al., Performance of mass spectrometric identification of clinical Prevotella species using the VITEK MS system: A prospective multi-center study. Anaerobe 54, 205-209 (2018).
46. M. Krzywinski, I. Birol, S. J. Jones, M. A. Marra, Hive plots-rational approach to visualizing networks. Brief Bioinform. 13, 627-644 (2012).
47. N. Takahashi, J. Washio, G. Mayanagi, Metabolomics of supragingival plaque and oral bacteria. J. Dent. Res. 89, 1383-1388 (2010).
48. E. Kanasi, et al., Clonal Analysis of the Microbiota of Severe Early Childhood Caries. Caries Res. 44, 485 (2010).
49. S. V. Cherkasov, et al., Oral microbiomes in children with asthma and dental caries. Oral Dis. 25, 898-910 (2019).
50. M. Freire, et al., Longitudinal Study of Oral Microbiome Variation in Twins. Sci. Rep. 10, 7954 (2020).
51. S. Nurk, D. Meleshko, A. Korobeynikov, P. A. Pevzner, metaSPAdes: a new versatile metagenomic assembler. Genome Res. 27, 824-834 (2017).
52. Y. W. Wu, B. A. Simmons, S. W. Singer, MaxBin 2.0: an automated binning algorithm to recover genomes from multiple metagenomic datasets. Bioinformatics 32, 605-607 (2016).
53. D. D. Kang, et al., MetaBAT 2: an adaptive binning algorithm for robust and efficient genome reconstruction from metagenome assemblies. Peer J7 (2019).
54. J. N. Nissen, et al., Improved metagenome binning and assembly using deep variational autoencoders. Nat. Biotechnol. 39, 555-560 (2021).
55. C. M. K. Sieber, et al., Recovery of genomes from metagenomes via a dereplication, aggregation and scoring strategy. Nat. Microbiol. 3, 836-843 (2018).
56. P. A. Chaumeil, A. J. Mussig, P. Hugenholtz, D. H. Parks, GTDB-Tk: a toolkit to classify genomes with the Genome Taxonomy Database. Bioinformatics 36, 1925-1927(2020).
57. D. H. Parks, M. Imelfort, C. T. Skennerton, P. Hugenholtz, G. W. Tyson, CheckM: assessing the quality of microbial genomes recovered from isolates, single cells, and metagenomes. Genome Res. 25, 1043-55 (2015).
58. R. M. Bowers, et al., Minimum information about a single amplified genome (MISAG) and a metagenome-assembled genome (MIMAG) of bacteria and archaea. Nat. Biotechnol. 35, 725-731 (2017).
59. C. T. Brown, et al., Unusual biology across a group comprising more than 15% of domain Bacteria. Nat. 523, 208-211 (2015).
60. J. Guo, et al., VirSorter2: a multi-classifier, expert-guided approach to detect diverse DNA and RNA viruses. Microbione 9, 1-13 (2021).
61. S. Nayfach, et al., CheckV assesses the quality and completeness of metagenome-1217 assembled viral genomes. Nat. Biotechnol. 39, 578-585 (2020).
62. S. Nayfach, Recommended cutoffs for analyzing CheckV results? BitBucket (2021) (Jul. 6, 2021).
63. C. Jain, L. M. Rodriguez-R, A. M. Phillippy, K. T. Konstantinidis, S. Aluru, High throughput ANI analysis of 90K prokaryotic genomes reveals clear species boundaries. Nat. Commun. 9, 1-8 (2018).
64. A. A. Hagberg, D. A. Schult, P. J. Swart, Exploring Network Structure, Dynamics, and Function using NetworkX (2008) (Jan. 11, 2018).
65. D. Hyatt, et al., Prodigal: prokaryotic gene recognition and translation initiation site identification. BMC Bioinformatics 11, 119 (2010).
66. B. Buchfink, C. Xie, D. H. Huson, Fast and sensitive protein alignment using DIAMOND. Nat. Methods 12, 59-60 (2014).
67. T. Aramaki, et al., KofamKOALA: KEGG Ortholog assignment based on profile HMM and adaptive score threshold. Bioinformatics 36, 2251-2252 (2020).
68. D. M. Emms, S. Kelly, OrthoFinder: phylogenetic orthology inference for comparative genomics. Genome Biol. 20, 1-14 (2019).
69. D. Kim, J. M. Paggi, C. Park, C. Bennett, S. L. Salzberg, Graph-based genome alignment and genotyping with HISAT2 and HISAT-genotype. Nat. Biotechnol. 37, 907-915 (2019).
70. Y. Liao, G. K. Smyth, W. Shi, featureCounts: an efficient general purpose program for assigning sequence reads to genomic features. Bioinformatics 30, 923-930(2014).
71. A. M. Bolger, M. Lohse, B. Usadel, Trimmomatic: a flexible trimmer for Illumina sequence data. Bioinformatics 30, 2114 (2014).
72. B. Langmead, S. L. Salzberg, Fast gapped-read alignment with Bowtie 2. Nat. Methods 9, 357 (2012).
73. E. A. Franzosa, et al., Species-level functional profiling of metagenomes and metatranscriptomes. Nat. Methods 15, 962-968 (2018).
74. F. Beghini, et al., Integrating taxonomic, functional, and strain-level profiling of diverse microbial communities with biobakery 3. Elife 10 (2021).
75. T. P. Quinn, I. Erb, Amalgams: data-driven amalgamation for the reference-free dimensionality reduction of zero-laden compositional data. 1-16 (2020).
76. J. L. Espinoza, ensemble_networkx: Ensemble networks in Python. GitHub (2020) (Jan. 22, 2021).
77. C. A. Ruiz-Perez, R. E. Conrad, K. T. Konstantinidis, MicrobeAnnotator: a user-friendly, comprehensive functional annotation pipeline for microbial genomes. BMC Bioinformatics 22, 1-16 (2021).
78. A. D. Fernandes, et al., Unifying the analysis of high-throughput sequencing datasets: Characterizing RNA-seq, 16S rRNA gene sequencing and selective growth experiments by compositional data analysis. Microbiome 2, 15 (2014).
79. T. P. Quinn, T. M. Crowley, M. F. Richardson, Benchmarking differential expression analysis tools for RNA-Seq: Normalization-based vs. log-ratio transformation-based methods. BMC Bioinformatics 19 (2018).
80. S. Mandal, et al., Analysis of composition of microbiomes: a novel method for studying microbial composition. Microb. col. Heal. Dis. 26 (2015).
81. Biocore, scikit-bio: A Bioinformatics Library for Data Scientists, Students, and Developers. GitHub (2020) (Jun. 4, 2020).
82. D. Opitz, R. Maclin, Popular Ensemble Methods: An Empirical Study. J. Artif Intell. Res. 11, 169-198 (1999).
83. M. Salibian-Barrera, R. H. Zamar, Bootstrapping Robust Estimates of Regression. Ann. Stat. 30, 556-582 (2002).
84. J. L. Espinoza, compositional: Compositional data analysis in Python. GitHub (2020) (May 15, 2020).
85. P. Virtanen, et al., SciPy 1.0: fundamental algorithms for scientific computing in Python. Nat. Methods 17, 261-272 (2020).
86. J. L. Espinoza, hive_networkx: Hive plots in Python. GitHub (2020) (Aug. 3, 2020).
87. J. L. Espinoza, soothsayer: High-level analysis package for (bio-)informatics. GitHub (2019) (Sep. 7, 2019).

1. A system for predicting dental caries in a subject, the system comprising:
a sequencing module configured to provide metatranscriptomic reads from an oral sample from a subject;
one or more processors in communication with the sequencing module; and
a memory in communication with the one or more processors, the memory storing instructions that, when executed by the one or more processors, cause the one or more processors to at least:
communicate with the sequencing module, such as sending instructions to transfer the metatranscriptomic reads from the sequencing module to the processor,
process instructions to read a metagenomic library from a database;
identify and cluster microbes identified in the oral sample into taxon clusters (TCs) using the metatranscriptomic reads mapped to the metagenomic library;
generate TC-specific orthogroups for each of the TCs via protein clustering;
determine KEGG orthology for each of the TC-specific orthogroups, or genes directly, using the metatranscriptomic reads mapped to the TC-specific orthogroups to provide KEGG modules for each of the TCs;
generate phylogenomic functional categories (PGFCs) from grouping of gene expression counts by the KEGG modules for each of the TCs;
determine a module completion ratio (MCR) for each of the KEGG modules in the PGFCs;
retain the PGFCs having the MCR above an MCR threshold to obtain input data; and
identify or predict, using a classifier model including variables selected by a feature selection machine learning algorithm, dental caries in said subject based on the input data,
further comprising generating a score based on an expression of genes from the metatranscriptomic reads as compared to genes within the KEGG module, and predicting or providing a diagnosis of dental caries in said subject when the score exceeds a threshold value, and
further comprising providing said subject a therapy for dental caries, such as removal of the dental caries, administration of dental fillings, providing a crown, root canal, or extraction.

2.-36. (canceled)
